(19) United States
(12) Patent Application Publication
(10) Pub. No.: US 2024/0175072 A1
(43) Pub. Date: May 30, 2024

(54) Method And Composition For Multiplexed And Multimodal Single Cell Analysis

(71) Applicants: Thermo Fisher Scientific Inc., Waltham, MA (US); Phitonex, Inc., Durham, NC (US)

(72) Inventors: Michael Stadnisky, Bend, OR (US); Craig LaBODA, Raleigh, NC (US); Nicholas PINKIN, Durham, NC (US); Ivan GODINEZ, San Diego, CA (US)

(21) Appl. No.: 18/254,135

(22) PCT Filed: Dec. 9, 2021
(86) PCT No.: PCT/US2021/062575

§ 371 Date: May 23, 2023

Related U.S. Application Data
(60) Provisional application No. 63/286,690, filed on Dec. 7, 2021.
(60) Provisional application No. 63/123,806, filed on Dec. 10, 2020.

(51) Int. Cl. C12Q 1/6804

(52) U.S. Cl. CPC C12Q 1/6804

(57) ABSTRACT

Provided herein are multimodal methods and compositions that combine sequence-tagged antibodies and fluorescent labels in a single reagent. Combined with optimal panel design, high-purity sorting of cells before sequencing has been demonstrated, and furthermore, truly quantitative information on the cell surface markers used for sorting.

### CROSS-REFERENCE TO RELATED APPLICATIONS

This application is a 35 U.S.C. 371 National Phase of PCT/US2021/062575 filed, Dec. 9, 2021, which claims the benefit under 35 U.S.C. § 119(e) of U.S. Provisional Application Nos. 63/286,690 filed Dec. 7, 2021, and 63/123,806, filed Dec. 10, 2020. The entire contents of the aforementioned applications are incorporated by reference herein.

### SEQUENCE LISTING

This application contains a Sequence Listing which has been submitted electronically in .txt format and is hereby incorporated by reference in its entirety. The material in the electronic Sequence Listing is submitted as an ASCII text (.txt) file entitled “LT01599PCT2-AS FILED-SL.txt” created on May 22, 2023, which has a file size of 4.00 KB (4,096 bytes). The Sequence Listing in this .txt file is part of the specification and is hereby incorporated by reference herein in its entirety.

### BACKGROUND

Currently, different reagents must be used to enrich/sort cells, e.g., through fluorescence-activated cell sorting, and analyze in deeper assays, e.g., through single-cell genomics. Similarly, different reagents must be used to image cells and cellular components and analyze in deeper assays, e.g., through single-cell genomic approaches. No methods exist, however, for bringing these modes of measurements together in one experiment or in individual samples. In other words, there is no single reagent suitable for multimodal use.

For ease of reading, this discussion of the current state of the art generally focuses on single-cell measurements and modalities as the single-cell is the fundamental unit of health and disease, but it is understood that the improvements disclosed in the Detailed Description that follows can also be applied to measurements taken on tissues (e.g., imaging) or bulk tissues (composed of single cells).

DNA barcoding (also called “feature barcoding”) is the process of attaching known DNA sequences to other molecules for later identification of each molecule using, for example, next generation sequencing (NGS) techniques. DNA barcodes can be used in single-cell analysis to uniquely identify individual cells from among many in a sample when performing gene sequencing. Additionally, DNA barcodes can be attached to antibodies that bind to cell surface receptors allowing observation of both genomic sequences within the cell and receptors on the cell surface using high throughput sequencing techniques.

Deterministic barcoding uses a specific known DNA sequence to tag a molecule, where the user knows precisely which DNA barcode is used to tag each molecule. In contrast, Stochastic barcoding relies on probability theory (Poisson statistics) to uniquely tag molecules of interest. For example, a user may desire to examine specific genes in individual cells across a sample of many cells. Instead of the labor-intensive task of specifically designing and tagging each gene of interest with this predetermined DNA sequence, the user relies on a large pool of known DNA sequences to stochastically (randomly) tag all the genes from a given cell with a DNA sequence. This approach requires the number of available DNA sequences to be much larger than the number of individual cells in the sample such that the probability of any DNA sequence associating with more than one cell is small.

There are many modalities of measurement of the genomic material in a single cell. Measurement of the Transcriptome (RNA) examines all of the genes expressed by an individual cell, and increasingly, the splicing status/isoforms of these RNA molecules themselves (e.g. estimated 10,000 protein coding genes in the human genome) through e.g.: Whole Transcriptome Analysis (WTA) which broadly analyzes individual cells, wherein a survey of all protein-coding genes is done or Targeted Sequencing in which a “panel” of genes is assayed, e.g., 400 genes with a focused approach (e.g. 400 gene immune panel, examining 400 genes involved in the immune response). Increasingly, these approaches can also be used to verify if there were any changes to a given gene, e.g., that occurred via gene editing (CRISPR). Measuring the Epigenome (DNA Accessibility and Chromatin) accounts that for an RNA to be transcribed, the gene, encoded in DNA, first needs to be accessible. This can be measured through various means, including assaying the DNA itself or by examining chromatin. There are various approaches to measuring this including CHIP-Seq and ATAC-Seq on either bulk or single cell samples.

There are many applications as well that aim to assay the Genome (DNA) including the extent of mutation, e.g., in cancer cells, recombination, e.g., in the case of T cell receptor or B cell receptor (done in bulk but can also be done for individual immune cells), gene editing, e.g., examining germline changes either due to CRISPR or other gene editing modalities (e.g. zinc finger nucleases) or gene addition/replacement/editing via gene therapy through various modalities.

Increasingly, multiple modalities including those above are combined to develop a comprehensive picture of individual cells from DNA to RNA and eventually, to protein.

Paramount to and alongside all of these different nucleic acid measurements is the single-cell Proteome. While all of the measurements above are genomic in nature, it is proteins that do the work of the cell and effect many various functions (e.g., interaction, enzymatic activity, communication, localization, stabilization, motility, etc.) on and within a cell. By definition, they are also the functional units wherein health and disease are caused and/or defined. Thus, while genomic measurements are important, arguably the most important is the Proteome. Proteins in or on the surface of the cell define both cell identity and also are the functional components of a cell. For instance, a memory T cell may be identified by its expression of CCR7 but this is also a chemokine receptor that functions to localize a memory T cell, e.g., into a particular part of the lymph node so it can perform its surveillance of incoming antigens and spring into action.

However, unlike the transcriptome, epigenome, and genome, which can be assayed using DNA-based primers and the toolbox of PCR (reverse transcriptase, heat, annealing, etc.) thanks to the complementarity of the bases of which each of these -omes is made, proteins require another assay modality, in which another protein (e.g. an antibody or variants thereof including but not limited to an aptamer, Fab fragment, etc.) specifically binds to an epitope of another protein. In the case of single cell genomics, these antibodies can be bound to barcodes (e.g., sequence-tagged antibody), which thus enable their detection in a sequencing assay (FIG. 1).

Single-cell transcriptome measurements suffer from the problem of “dropouts.” As most RNA molecules are present at single digit copies, except for very highly expressed genes like housekeeping genes, it is critically important to have greater depth of sequencing to ensure that a zero that is detected is a “true” zero. Sequencing depth (a.k.a. coverage) is the number of unique reads that include a given nucleotide in the reconstructed sequence. RNA Sequencing generally requires greater depth. This problem is made worse by the fact that immune cells express very low copies of RNA and sometimes zero copies of RNA of proteins for which they are defined. For example, a CD4+ Helper T cell which is defined by having the CD4 co-receptor on its surface generally expresses zero copies of the CD4 gene in RNA. Thus, assaying cells, especially immune cells, requires deeper sequencing (i.e. more reads), which drives costs higher.

Sequencing depth enables examination of more features of an individual cell. Importantly, in a discovery experiment, it is very often completely unknown if more sequencing will yield the discovery of more features. Generally, this leads to stepwise experiments, with WTA run on an enriched cell population followed increasingly by running a targeted panel of genes with more depth. As certain cell types are quite rare, running more cells or running more sequencing runs (for additional depth, e.g., in the detection of RNA isoforms or post-transcriptional processing) has a dramatic effect on the cost per cell (world wide web at satijalab.org/costpercell) and thus puts a downward pressure on the number of cells per experiment. In fact, based on the current standard of a “$1000 genome” (which really means ˜1.5× coverage CNV), the cost of any sequence-tagged antibody experiment scales linearly and well beyond the cost of a flow cytometry experiment. In turn, at the current “$1000 genome stage,” sorting for cell enrichment is absolutely critical, most rare cell analysis must still be done by flow cytometry, and the application of single-cell technologies with sequence-tagged antibodies has a long road to being used in clinical diagnostics.

To that end, with so much pressure on discoveries of more specific cell phenotypes and rarer cells driving sequencing cost higher and higher, scientists almost always enrich a cell population of interest before they use those single cells in a downstream -omics assay. This can be accomplished in two different ways or a combination thereof. First, magnetic (or bubble) enrichment, in which positive or negative enrichment can be performed using commercially available metal particles or microbubbles conjugated to antibodies. Second, sorting (fluorescence-activated cell sorting, FACS), in which the majority of cell enrichment before any single cell experiment uses FACS and fluorophore-tagged antibodies to sort cell populations of interest for downstream analysis.

The final significant cost driver is the cost of the sequence-tagged antibodies themselves, which are sold at very high average sale prices, and must often be used in combination with fluorescent tagged antibodies to perform the sorting. FIG. 2 shows a currently available single-cell sequencing workflow with sequence-tagged antibodies.

Single-cell sequencing has enabled an explosion of parameters measured per cell from droplet-based methods that can be used to examine the whole transcriptome (WTA, i.e., every RNA) of a cell, to multi-modal measurements. That being said, there are several known approaches to single-cell “compartmentalization” or isolation methods, representative examples of which are shown in Table 1.

TABLE 1

| Method | Concise Description | Reference |
|---|---|---|
| Fluidigm C1 | Microfluidic single cell | World wide web at: |
| | isolation | www.fluidigm.com/products/c1-system |
| SeqWell | subnanoliter wells, DNA | Gierahn, T., Wadsworth, M., Hughes, T. et al. |
| | library prep kit | Seq-Well: portable, low-cost RNA sequencing |
| | | of single cells at high throughput. Nat Methods |
| | | 14, 395-398 (2017). |
| | | https://doi.org/10.1038/nmeth.4179 |
| CellSee | microfluidic gravity-based | World wide web at: www.celsee.com |
| | approach. Discrete | |
| | microwells | |
| DropSeq | cell sorting/droplet | Macosko et al. Highly Parallel Genome-wide |
| | capture/lysis, rna capture in | Expression Profiling of Individual Cells Using |
| | library | Nanoliter Droplets Cell. 2015. |
| ddSeq | digital droplet PCR | World wide web at: info.bio-rad.com/ww- |
| | adapted for cell isolation | ddseq.html?WT.mc_id=170714020574&WT.s |
| | | rch=1&WT.knsh_id=5684c912-a659-4fa4- |
| | | bfbc- |
| | | bc2ccf8ec9b1&gclid=Cj0KCQjws536BRDTA |
| | | RIsANeUZ58L6CT8V782Hwdez- |
| | | 4X8cRsMdu47fcKM18i6pupbXxsmX- |
| | | VUEzSA2gaAixREALw_wcB |
| SCISeq | well-based | Vitak et. al. SCI-seq: Sequencing thousands of |
| | | single-cell genomes with |
| | | combinatorial indexing. Nature Methods. |
| | | 2017. |
| 10X Chromium* | cell sorting/droplet | World wide web at: www.10xgenomics.com |
| | capture/lysis, dna capture | |
| | in library | |
| BD Rhapsody* | microwell based | World wide web at: go.bd.com/bd- |
| | | rhapsody.htm |
| Mission Bio Tapestri | cell sorting/droplet | |
| | capture/lysis, dna capture | |
| | in library | |

Star (*) indicates that sequence-tagged antibodies have been used in combination with these technologies.

Further there are several approaches to single-cell measurements, representative examples of which are shown in Table 2.

TABLE 2

| Method | Concise Description |
|---|---|
| Transcriptome* | measuring copies of mRNA either across the entire transcriptome |
| | (WTA) or using targeted panels (examining 100s of selected |
| | genes) |
| | SMART-Seq/SMART-Seq2 for improved read coverage |
| | allowing the detection of alternative transcript isoforms and SNPs |
| TCR/BCR sequencing* | DNA sequencing to examine the T and/or B cell receptors, which are |
| | generated through random rearrangement of genomic and determine the |
| | specificity of these cells |
| DNA Seq* | Single Cell CNV |
| Sequence-tagged | CITESeq |
| antibodies | TotalSeq (“proteogenomics”) |
| | AbSeq |
| DNA accessibility* | single cell ATAC-Seq |
| Gene editing* | ECITE-Seq |

Star (*) indicates that sequence-tagged antibodies have been used in combination with these technologies.

Generally, immunofluorescence (IF) imaging is the process by which proteins of interest can be detected using either primary antibodies covalently conjugated to fluorophores (direct detection) or a two-step approach with unlabeled primary antibody followed by fluorophore-conjugated secondary antibody (indirect detection). Either method allows the user to combine multiple fluorophores (multiplex analysis), making IF ideal for investigating protein co-localization, changes in subcellular localization, differential activation of proteins within a cell, identification of different cell subsets, and other analyses. Critically, massively multiplexed modalities have been created leveraging genomic material as the “velcro” for staining, or genomic assays have been developed which can be used in combination with imaging to get both phenotypic and functional information about a cell (imaging) and the component gene expression of these cells (or regions). This brings the addition of location-based data that can show how cells and tissues are organized and visualize cell-cell interactions. For example, activated cytotoxic CD8+ T cells specific for a given tumor antigen could be present in a tumor tissue—single-cell methods of measurement including flow cytometry and those listed in Table 1 and Table 2 above would show the presence of these cells. However, they could be physically occluded from the tumor, rendering them useless. Thus, one generally trades throughput (cell number) for gaining the additional insight of cell location. Table 3 shows various representative tissue imaging modalities used in life sciences.

TABLE 3

| Method | Concise Description | Challenges |
|---|---|---|
| Traditional | low plex imaging, often | high background due to |
| immunofluorescence | leveraging secondary | various tissue processing |
| imaging | antibodies for signal | steps, antigen retrieval, and |
| | amplification | use of secondary antibodies |
| | can include protein | low plex due to use of |
| | measurements either through | traditional fluorescent dyes |
| | labeling with specific | and limitations of secondary |
| | molecules, dyes that stain | antibody multiplexing |
| | cell components, labeling | not possible/very, very |
| | individual proteins, e.g. | challenging to do combined |
| | GFP/RFP, | genomic - proteomic |
| | also genomic measurements, | measurement |
| | e.g. FISH or MERFISH | |
| | (among many others) | |
| Mass Spec methods | mass-spec and tissue- | destroys the tissue so |
| (IMC ™ or MIBI ™) | destructive methods using | further analysis is not |
| | mass labels to perform | possible |
| | multiplexed tissue imaging | capex and dedicated |
| | | operator instruments set a |
| | | higher barrier to entry |
| | | not possible to do combined |
| | | genomic - proteomic |
| | | measurement |
| CODEX ® from Akoya | sequence-tagged antibodies | requires amplification step |
| | used and combined with | not possible to do combined |
| | dye-labeled reporters | genomic - proteomic |
| | will function to a higher plex | measurement |
| | through multiple rounds of | |
| | staining and can be | |
| | integrated with OpalTM to | |
| | streamline workflows | |
| Spatial transcriptomics | can now be combined with | regions rather than |
| (20+ approaches here | traditional | individual cell resolution |
| include Visium from | immunofluorescence | with combined workflow, |
| 10X Genomics) | imaging | can do combined genomic - |
| | | proteomic measurement |
| | | with caveat that it's cells in |
| | | imaging and regions in |
| | | transcriptomic |
| | | Protein and RNA are |
| | | assayed independently and |
| | | the RNA is only sampled in |
| | | sections. |
| Ultivue's InSituPlex ® | antibodies against four | low plex |
| | different targets are added to | pre-set panels |
| | the sample simultaneously | requires amplification |
| | and the conjugated oligos | not possible to do combined |
| | are subsequently amplified | genomic - proteomic |
| | target detection requires the | measurement |
| | addition of fluorescently | |
| | labeled complementary | |
| | DNA probes | |
| Sequential Staining | Complicated sequential | harsh chemistry |
| (MultiOmyx ™, Opal ™) | fluorescent staining of | complicated workflows |
| | tissues using traditional | heat inactivation |
| | fluorescent dye-tagged | not possible to do combined |
| | antibodies and chemistry to | genomic - proteomic |
| | strip | measurement |

FIG. 3 shows a representative current workflow for combining immunofluorescence imaging and gene expression. In the outlined workflow, analysis of protein and the whole transcriptome on tissue sections (or whole tissues) are done at independent steps, but the workflow integrates with current histological laboratory methods and tools for tissue analysis.

There exists a need, however, for a reagent and methods to combine various modalities and/or workflows or preserve the optionality following a measurement to make a decision about the ensuing analysis to pursue.

### SUMMARY

Provided for herein are methods for combining cell enrichment, cell sorting, and/or immunofluorescent cell labeling with genomic analysis using a sequence-tagged fluorescent-label specificity determining molecule conjugate comprising both a fluorescent label component and a specificity determining molecule component, wherein one or more components of the conjugate are used for cell enrichment, cell sorting, and/or immunofluorescent cell labeling and one or more components of the same conjugate are utilized in the genomic analysis. The methods provided herein comprise (a) performing cell enrichment, cell sorting, and/or immunofluorescent cell labeling on a cell and/or sample of cells and (b) performing genomic analysis on the same cell and/or sample of cells, using the fluorescent-labeled sequence-tagged specificity determining molecule conjugate. In certain embodiments, the specificity determining molecule component is sequence-tagged. In certain embodiments, the method first comprises contacting the cell and/or sample of cells with the fluorescent-labeled sequence-tagged specificity determining molecule conjugate. And, in certain embodiments, the genomic analysis occurs after the cell enrichment, cell sorting, and/or immunofluorescent cell labeling.

Also provided for herein is a sequence-tagged fluorescent-label specificity determining molecule conjugate comprising a specificity determining molecule component conjugated to a fluorescent label component, wherein said conjugate is suitable for use in one or more of the methods of this disclosure. In certain embodiments, the specificity determining molecule component is sequence-tagged. In certain embodiments, the fluorescent label component is attached to the specificity determining molecule component via a nucleic acid linker, wherein the nucleic acid linker comprises a double-stranded segment. In certain embodiments, the nucleic acid linker is entirely double-stranded. In certain embodiments, the nucleic acid linker is from any of about 10, 15, 20, 25, 30, 35, 40, 45, 50, 55, 60, 65, 70 or 75 nucleotides in length to any of about 15, 20, 25, 30, 35, 40, 45, 50, 55, 60, 65, 70, 75, 80, 85, 90, 95 or 100 nucleotides in length. In certain embodiments, the nucleic acid linker is double-stranded and is between 30 and 70 nucleotides. In certain embodiments, the specificity determining molecule component comprises a PCR primer region, a barcode region and a capture sequence. In certain embodiments, the specificity determining molecule component further comprises an oligonucleotide sequence for attachment of the fluorescent label component. In certain embodiments the fluorescent label component is a genomic fluor. In certain embodiments, the genomic fluor is a multimodal label comprising a fluorescent moiety and a unique identifying sequence.

Certain embodiments are directed to a kit for performing a method of this disclosure.

Certain embodiments are directed to a method of validating a sequence-tagged antibody comprising contacting a genomic fluor comprising a nucleic acid linker with a sequence-tagged antibody and running a sample by flow cytometry to evaluate the antibody's binding to its target.

Provided for herein is a method of tuning the brightness of a polynucleotide-modified biomolecule bioconjugate of the present disclosure, the method comprising i) altering the total length of the nucleic acid linker, ii) altering the length of the fully double-stranded region of the nucleic acid linker, iii) altering the length of the single-stranded portion of the nucleic acid linker, and/or iv) having the single-stranded portion comprise a poly(A), poly(T), poly(G), poly(C) sequence and/or a unique nucleic acid sequence.

### BRIEF DESCRIPTION OF THE DRAWINGS/FIGURES

FIG. 1 shows a representation of a sequence-tagged antibody.

FIG. 2 shows a single-cell sequencing workflow with sequence-tagged antibodies.

FIG. 3 shows a representative current workflow for combining immunofluorescence imaging and gene expression.

FIG. 4A shows use of a hybridized double-stranded linker sequence, e.g., poly(A)/poly(T) in a sequence-tagged fluorescent-label specificity determining molecule according to an embodiment of the present disclosure. FIG. 4B shows another example of a composition according to the present disclosure comprising a sequence-tagged antibody with an attached sequence comprising a unique identifying sequence with a primer sequence for amplification, a capture sequence, which can include a single stranded poly(A) linker sequence, and an additional oligonucleotide sequence (OTdN) to which a single-stranded linker sequence is attached that can hybridize to a complementary linker sequence attached to a fluorescently labeled nucleic acid nanostructure. The sequence of the nucleic acid linker that links the nucleic acid nanostructure to the sequence tag can vary and can be either a unique sequence or a repeated sequence (e.g., a poly(A), poly(T), poly(C) or poly(G) sequence).

FIGS. 5A and 5B show a direct linkage between an antibody and a nucleic acid nanostructure using a nucleic acid linker.

FIG. 6 illustrates issues with current commercially available sequence-tagged antibodies, which can be examined and revealed using the methods described herein. In this example, two oligo-modified versions of the same antibody and clone were labeled with the same genomic fluor. In one case, the modified antibody's performance has been degraded and it does not bind to its target, resulting in only a single, negative population. In the other case, the modified antibody retains its activity, targeting the antigen, and separating out a positive population.

FIGS. 7A-7D illustrate four different expression levels that can be encountered in single cells and populations of cells. FIG. 7A demonstrates that cells with low expressing genes, proteins, or other biological molecules, individual cells will have a distribution of expression, and due to drop-outs in NGS, it is impossible to determine if a cell has a “real” zero measurement and thus more (deeper) sequencing (i.e., more reads and higher cost) are used, as well as imputation (informatics approach to assign a non-zero “probability of expression”, e.g., Badsha, M. B., Li, R., Liu, B. et al. Imputation of single-cell gene expression with an autoencoder neural network. Quant Biol 8, 78-94 (2020). https://doi.org/10.1007/s40484-019-0192-7.) to establish “true” zero's or a probability of expression in the current state of the art. FIG. 7B shows the high end of the expression range, at which several key identifying proteins may be for an individual cell or housekeeping gene expression may exist, on which a preponderance of sequencing and thus cost is spent measuring. FIG. 7C shows an example expression range, e.g., for measuring gene expression of interferon gamma gene expression (ifng) on an immune cell population (CD4+ helper T cells). FIG. 7D shows a theoretical example wherein both measurements are brought closer to the same dynamic range and thus receive near-equal “sequencing” weight as measured by number of reads.

FIG. 8 illustrates “epitope blocking.” Once a fluorescent dye conjugated antibody is bound to an epitope on a protein, an antibody that recognizes the same epitope or nearby epitope will be blocked from subsequently binding.

FIG. 9A illustrates a modified one-step labeling workflow enabled by the compositions and methods of this disclosure. FIG. 9B illustrates a workflow for obtaining cell surface protein and transcript data from individual cells according to certain methods of the present disclosure. Briefly, cells (e.g., peripheral blood mononuclear cells (PBMCs) are stained in suspension with the fluorescently labeled sequence-tagged antibodies provided herein to delineate major immune cell types; cells undergo fluorescence-activated cell sorting (FACS) to select for cells of interest; enriched cells are processed through an scRNAseq workflow; and resulting data provides researchers the ability to obtain protein and transcript data, enabling deeper insights into complex biological systems.

FIG. 10A shows sequential staining for imaging workflow. In certain embodiments, amplification can be added as described elsewhere herein, but is not necessary for imaging. FIG. 10B shows a workflow for obtaining spatial proteogenomics data according to certain methods of the present disclosure. Stored tissue blocks (either formalin-fixed paraffin-embedded (FFPE) or formalin-fixed (FF)) are prepared using standard protocols. Briefly, using a microtome, slice the tissue and mount onto a charged slide, perform antigen retrieval, stain tissue samples with the fluorescently labeled sequence-tagged antibodies provided herein, proceed to desired downstream imaging and processing, and perform data analysis.

FIG. 11A shows antibodies and fluorescent nucleic acid nanostructures (e.g., PHITON nucleic acid nanostructures) that were modified with varying lengths of ssDNA linkers that completely or partially hybridized to one another. FIG. 11B shows the various antibody-fluorescent nucleic acid nanostructure conjugates using different combinations of the individual components shown in FIG. 11A. FIG. 11C shows a polyacrylamide gel electrophoresis (PAGE) gel showing antibody-ssDNA linker conjugates for each of the four lengths of ssDNA linker on the antibody (16, 32, 69, 100 nucleotides) after purification to remove unmodified antibody.

FIG. 12A shows flow cytometry data from human PBMCs testing the various possible combinations of nucleic acid linkers for attaching a fluorescent nucleic acid nanostructure (in this example NOVAFLUOR Yellow 610) to anti-Human CD4 antibody (clone SK3). All conjugates were compared at the same dose. FIGS. 12B and 12C show analysis of the flow cytometry data comparing the median fluorescence intensity (MFI) of the CD4+ population and the separation indices (SI) of the various antibody-NOVAFLUOR Yellow 610 conjugates. The composition of the nucleic acid linker strongly influenced the performance of the conjugate in flow cytometry. FIG. 12D shows the composition of the nucleic acid linkers for each of the conjugates, specifically whether the linker was partially or fully double-stranded and whether the single-stranded portion of the nucleic acid linker contained a poly(T) region and/or a unique identifying sequence (UNIQ).

FIG. 13A shows anti-human CD4 antibody (clone SK3) conjugated to NOVAFLUOR Yellow 570 and anti-human CD8 antibody (clone OKT-8) conjugated to NOVAFLUOR Yellow 660 using two different nucleic acid linker sequences (Poly(A)/Poly(T) for the CD4 conjugate and a more varied sequence “varied linker” for the CD8 conjugate). FIG. 13B shows flow cytometry data showing co-staining of the CD4 and CD8 conjugates described in FIG. 13A on PBMCs.

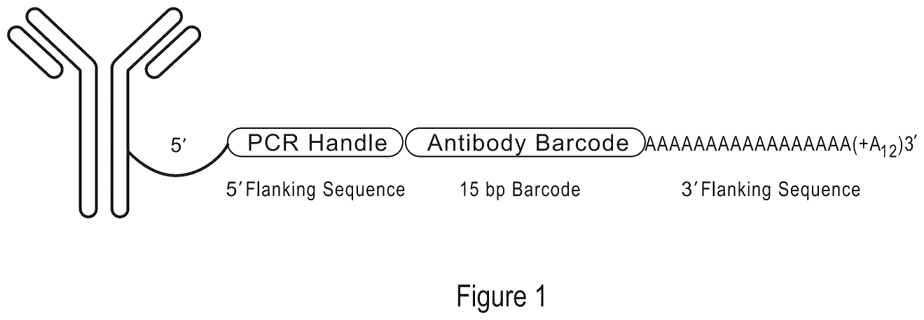

### DETAILED DESCRIPTION

### Definitions

It is to be noted that the term “a” or “an” entity refers to one or more of that entity; for example, “a linker,” is understood to represent one or more linkers. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising” or “comprises” otherwise analogous aspects described in terms of “consisting of,” “consists of,” “consisting essentially of,” and/or “consists essentially of,” and the like are also provided.

Unless defined otherwise, technical and scientific terms used herein have the same meaning as commonly understood by one of ordinary skill in the art to which this disclosure is related.

Numeric ranges are inclusive of the numbers defining the range. Even when not explicitly identified by “and any range in between,” or the like, where a list of values is recited, e.g., 1, 2, 3, or 4, unless otherwise stated, the disclosure specifically includes any range in between the values, inclusive of the end-points, e.g., 1 to 3, 1 to 4, 2 to 4, etc.

The headings provided herein are solely for ease of reference and are not limitations of the various embodiments or aspects of the disclosure, which can be had by reference to the specification as a whole.

As used herein, a “linker” is a component of a conjugated molecule whose purpose is to link together other components of the molecule or, when the other components of the conjugated molecule are not linked together, the portion of a component present for the purpose of conjugating to another constituent but that would otherwise not necessarily be present. For example, an antibody would not normally or necessarily have a polynucleotide attached to it, but for the purposes of this disclosure, a polynucleotide can be attached to an antibody to form a linker to link the antibody to another molecule to form a conjugate molecule. Likewise, a nucleic acid nanostructure of this disclosure may not necessarily have a certain at least partially single-stranded extension, but for the purposes of this disclosure, a nucleic acid nanostructure can comprise an at least partially single-stranded linker extension to link the nanostructure to another molecule, such as an antibody, to form a conjugate molecule.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retains at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, deletions or additions. Derivatives are polypeptides that have been altered so as to exhibit additional features not found on the native polypeptide. Examples include fusion proteins. Derivative polypeptides can also be referred to herein as “polypeptide analogs.” As used herein a “derivative” can refer to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

As used herein, the term “specificity determining molecule” refers in its broadest sense to a molecule that recognizes a target molecule (target) and associates with it. Specificity determining molecules include binding molecules that can specifically bind to an antigenic determinant, such as an antibody binds an epitope, and also molecules that can bind to receptors, such as receptor ligands (e.g., gastrin-releasing peptide (GRP) and gastrin-releasing peptide receptor (GRPR)). Thus, representative examples of specificity determining molecules include peptides, recombinant, natural, or engineered receptor/ligand proteins, aptamers, tetramers (folded MHC proteins with peptides used for detecting T cell receptors), non-antibody proteins or antibody mimetics, e.g., affilins, affimers, affitins, alphabodies, avimers, fynomers, Kunitz domain peptides, nanoCLAMPS, Designed Ankyrin Repeat Proteins (DARPins), monobodies, anticalins, affibodies, and SOMAmers (further examples are referred to in the Global Bioanalysis Consortium (GBC) and the European Medicines Agency “classification of critical reagents as analyte specific or binding reagents, specifically antibodies; peptides; engineered proteins; antibody, protein and peptide conjugates; reagent drugs; aptamers and anti-drug antibody (ADA) reagents including positive and negative controls (King, L E, et al. Ligand Binding Assay Critical Reagents and Their Stability: Recommendations and Best Practices from the Global Bioanalysis Consortium Harmonization Team. AAPS J. 2014 May; 16(3): 504-515). In certain embodiments, a specificity determining molecule may target genomic material, e.g. DNA or RNA, to perform FISH or other biological assays, e.g., on chromatin accessibility or gene expression.

Disclosed herein are certain binding molecules comprising antibodies, or antigen-binding fragments, variants, or derivatives thereof. Unless specifically referring to full-sized antibodies such as naturally-occurring antibodies, the term “binding molecule” encompasses full-sized antibodies including bispecific antibodies (e.g., comprising a first binding domain binding to a first epitope, and a second binding domain binding to a second epitope), as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

The terms “antibody” and “immunoglobulin” can be used interchangeably herein. Basic immunoglobulin structures in vertebrate systems are relatively well understood. See, e.g., Harlow et al., Antibodies: A Laboratory Manual, (Cold Spring Harbor Laboratory Press, 2nd ed. 1988). Antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “chimeric antibody” will be held to mean any antibody wherein the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The term “bispecific antibody” as used herein refers to an antibody that has binding sites for two different antigens within a single antibody molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which the variable domain in either the heavy and light chain or both is altered by at least partial replacement of one or more CDRs from an antibody of known specificity and, by partial framework region replacement and sequence changing. Although the CDRs can be derived from an antibody of the same class or even subclass as the antibody from which the framework regions are derived, it is envisaged that the CDRs will be derived from an antibody of different class, e.g., from an antibody from a different species. An engineered antibody in which one or more “donor” CDRs from a non-human antibody of known specificity is grafted into a human heavy or light chain framework region is referred to herein as a “humanized antibody.” In some instances, not all of the CDRs are replaced with the complete CDRs from the donor variable region to transfer the antigen binding capacity of one variable domain to another; instead, minimal amino acids that maintain the activity of the target-binding site are transferred. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

The term “polynucleotide” (also referred to as an “oligonucleotide”) is intended to encompass a singular nucleic acid as well as plural nucleic acids with “nucleic acid” referring to, for example, DNA or RNA or an analog thereof such as comprising a synthetic backbone or base. In certain embodiments, the polynucleotide or nucleic acid is DNA. In other embodiments, a polynucleotide or nucleic acid can be RNA. A nucleic acid or polynucleotide can comprise a conventional phosphodiester bond or a non-conventional bond (e.g., an amide bond, such as found in peptide nucleic acids (PNA)). By “isolated” nucleic acid or polynucleotide is intended a nucleic acid molecule, DNA or RNA, which has been removed from its native environment such as an isolated nucleic acid molecule or construct, e.g., messenger RNA (mRNA) or plasmid DNA (pDNA). For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, a polynucleotide or a nucleic acid can be or can include a regulatory element such as a promoter, ribosome binding site, or a transcription terminator.

A “nucleic acid nanostructure” is an oligonucleotide construction of any size and composed of one or more oligonucleotide strands and can have a tertiary and/or a quaternary structure and be composed of natural and/or synthetic nucleic acid bases. A nucleic acid nanostructure comprised substantially or entirely of DNA is also referred to herein as a DNA nanostructure. In certain embodiments, a nucleic acid nanostructure can include fluorescent moieties of any type, including but limited to small organic dyes (of all varieties and base structures, e.g. rhodamines, cyanines, oxazines, etc.), naturally occurring fluorophores, phosphorescent molecules, fluorescent proteins, fluorescent polymers, quantum dots or other fluorescent nanoparticles, upconverting particles, lanthanides, and bioluminescent molecules, as well as unique identifying sequences. As used herein, the terms “nucleic acid nanostructure” and “nanostructure” are interchangeable. A fluorescently labeled nucleic acid nanostructure is also referred to herein as a “genomic fluor”.

As used herein, a “PHITON” (Thermo Fisher Scientific, Waltham, MA) is a nucleic acid nanostructure produced by PHITONEX, Inc. (now a part of Thermo Fisher Scientific), Durham, North Carolina (U.S. Patent Publication No. 2020/0124532, Lebeck, A., Dwyer, C., LaBoda C. Resonator Networks for Improved Label Detection, Computation, Analyte Sensing, and Tunable Random Number Generation; which is incorporated herein in its entirety). PHITON nucleic acid nanostructures are fluorescent labels composed of a DNA-based scaffold that precisely arranges fluorophores in order to engineer their interactions and the overall fluorescent properties of the structure. The underlying scaffold presents many unique opportunities for fluorescence amplification. For example, as disclosed herein, the underlying scaffold can be leveraged to programmatically control the interactions between individual PHITONs in order to chain them together for a collectively enhanced fluorescence signal. Examples of PHITON nucleic acid nanostructures are NOVAFLUOR nucleic acid nanostructures (Thermo Fisher Scientific, Waltham, MA).

As used herein, unless otherwise specified, “complementary base pairing” refers to A/T, A/U, or C/G base pairing and corresponding pairing of synthetic or non-standard nucleotides, e.g., isocytosine/isoguanine (isoC/isoG). To the extent that thymidine (T) is specified as a base in a nucleic acid, for the purposes of simplifying this disclosure, unless otherwise specified, it is understood that uracil (U) is intended if the nucleic acid is RNA.

Unless otherwise specified in a particular context, the terms “conjugated to” and “linked to” are used interchangeably herein.

As used herein, a “conjugate” is a composition having distinct parts, components, moieties, constituents, or the like linked together.

As used herein, “cell enrichment” modalities include magnetic or bubble-based enrichment including positive or negative enrichment via metal particles or microbubbles conjugated to specificity determining molecules and microfluidic-based cell enrichment based on size or other characteristics e.g., fluorophore-conjugated specificity determining molecules; or a combination of one or more these methods (generally the concept is enriching either positively or negatively based on cell characteristics like identity, size, granularity, mass, etc.).

“Cell sorting” modalities such as fluorescence-activated cell sorting (FACS) include the use of fluorophore-conjugated specificity determining molecules to sort/enrich cell population(s) of interest, e.g., for downstream analysis.

As used herein, “immunofluorescent cell labeling” modalities involve the process in which antigens (such as protein antigens) of interest that are expressed in or on a cell can be detected using primary antibodies covalently conjugated to fluorophores (direct detection), a two-step approach with unlabeled primary antibody followed by fluorophore-conjugated secondary antibody (indirect detection), or other variations known to those of skill in the art. Additionally, such methods can include the use of cell membrane or DNA stains. In this manner, one or a multitude of cells from one or more samples, tissues, patients, etc., can be measured via immunofluorescent techniques (flow cytometry, immunofluorescence imaging, etc.) and/or enriched with techniques such as FACS.

As used herein, “genomic analysis” modalities involve the examination of the transcriptome (e.g., identity, copy number of mRNA or other RNA species including alternative transcript isoforms and single nucleotide polymorphisms (SNPs)). Representative examples include using whole transcriptome analysis (WTA) or using targeted panels (e.g., examining 100s of selected genes), on a per-cell or per-tissue basis, as well as potentially determining the location of the RNA in combination with its identity); T and or B cell receptor sequencing in which DNA sequencing is performed to examine the receptors of these immune cells; DNA sequencing to examine germline DNA e.g., to detect copy-number variation (CNV) at a single cell level; the use of sequence-tagged antibodies to examine protein/antigen expression through methods such as CITESeq, TotalSeq (“proteogenomics”) or AbSeq; assessing DNA accessibility and chromatin e.g., through single cell ATAC-Seq; assessing the extent and targets of gene editing e.g., through single cell CRISPR screens; or a combination of one or more of the methods listed above. In addition to cells in suspension-based methods, genomic analysis also includes the addition of location-based data either through assaying genomic material directly e.g., FISH, MERFISH, spatial transcriptomics or by leveraging a sequence tag to assay the presence and location of proteins and other antigens e.g., through the use of sequence-tagged antibodies. The measurement of either in-solution or location-based assays could include the use of Sanger sequencing, next-generation sequencing (NGS), long read sequencing, or in situ sequencing.

As used herein, a “fluorescent label” (also called a fluorophore, fluorescent tag, fluorescent dye, or fluorescent probe) is a molecule that is attached to aid in the detection of a biomolecule such as a protein, antibody, or polynucleotide. A fluorescent label may be a naturally occurring fluorescent protein (e.g. phycoerythrin, PE), a derivative thereof (e.g. PE-Cy7) including tandem dyes, polymer dyes, single molecule dyes, fluorescent nucleic acids, or scaffold-based fluorescent labels e.g. nucleic acid nanostructures including fluorescent DNA nanostructures.

Unless otherwise specified, the terms “barcode,” “feature barcode,” and “unique identifying sequence” are used interchangeably and refer to an oligonucleotide sequence that can be used to distinguish between one or multiple species.

### Overview

Currently there is no good way to combine fluorescent based detection and genomic analysis workflows or preserve the optionality following a measurement to make a decision about the ensuing analysis to pursue. There is also no link to the central canon of biology (cell phenotypes and function). Further, epitope/antigen blocking and validation is impossible in current paradigms and costs are high because antibodies must be duplicated to analyze a single target.

Provided herein is a novel marriage of components for different modalities, for example, combining sequence-tagged antibodies and fluorescent labels. For purposes of a sequence-tagged antibody of this disclosure, an “antibody” is a type of specificity determining molecule, either naturally occurring or synthetic. A specificity determining molecule may be a protein, enzyme, and/or substrate, which enables the assaying of multiple modes/-omes using the embodiments described herein. In certain embodiments, a specificity determining molecule may target genomic material, e.g. DNA or RNA, to perform FISH or other biological assays, e.g., on chromatin accessibility or gene expression. One of the advantages of a single specificity determining molecule that has both a sequence tag component and a fluorescent label component is that both fluorescent-based detection and genomic analysis can be analyzed using the same specificity determining molecule.

The present disclosure is not limited to any particular detection modality. While illustrative examples include next-generation sequencing, immunofluorescence imaging, and flow cytometry/FACS, it is understood that multiple genomic detection methods (including those that amplify) and fluorescence read-out measurement modalities are useful and contemplated.

One of ordinary skill in the art will recognize without constant repeating that when a nucleic acid linker of an element (such as a specificity determining molecule or nucleic acid nanostructure) is intended to hybridize with a nucleic acid linker of another element, the single-stranded portion of each linker is sufficient in length, complementarity, and continuity to allow for hybridization.

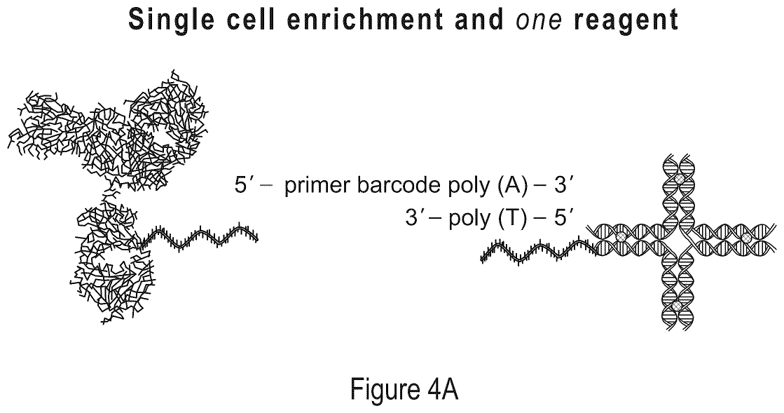

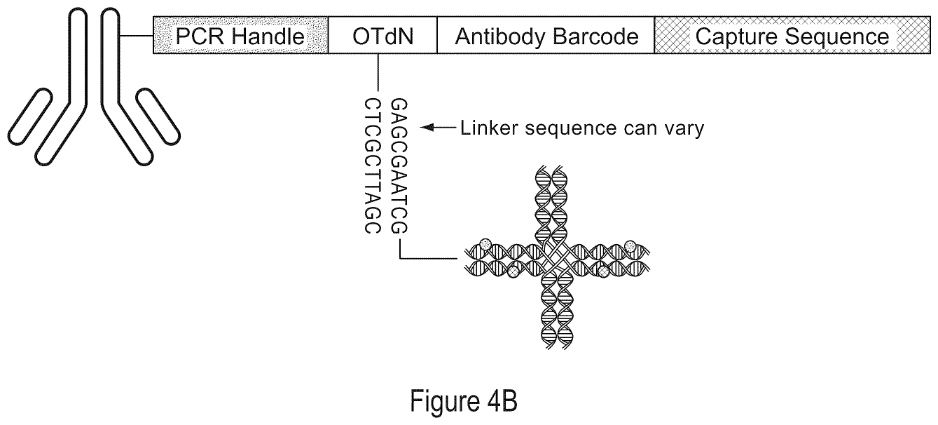

Provided for herein is a composition such as an assay reagent comprising a specificity determining molecule (such as an antibody), a fluorescent label (such as a fluorescent nucleic acid nanostructure), and a unique identifying oligonucleotide sequence, enabling single and multiple read-outs. That is, for performing either an individual measurement, e.g. single-cell protein measurement, or for enabling the optionality of performing another experiment as part of a workflow. FIG. 4A shows a composition comprising a sequence-tagged antibody with an attached sequence comprising a unique identifying sequence with a primer sequence for amplification and a single-stranded poly(A) linker sequence hybridized to a complementary poly(T) linker sequence attached to a fluorescently labeled nucleic acid nanostructure, wherein the sequence-tagged antibody with the unique identifying sequence and the fluorescently labeled nucleic acid nanostructure are indirectly linked together (i.e., no direct covalent attachment) via the hybridized double-stranded poly(A)/poly(T) linker sequence. FIG. 4B shows another example of a composition according to the present disclosure comprising a sequence-tagged antibody with an attached sequence comprising a unique identifying sequence with a primer sequence for amplification, a capture sequence, which can include a single stranded poly(A) linker sequence, and an additional oligonucleotide sequence (OTdN) to which a single-stranded linker sequence is attached that can hybridize to a complementary linker sequence attached to a fluorescently labeled nucleic acid nanostructure. The sequence of the nucleic acid linker that links the nucleic acid nanostructure to the sequence tag can vary and can be either a unique sequence or a repeated sequence (e.g., a poly(A), poly(T), poly(C) or poly(G) sequence). The same composition can be used, for example, in both flow cytometry and sequence-tagged antibody modalities. In certain embodiments, a nucleic acid linker linking a specificity determining molecule component to a fluorescent label component, unless otherwise stated, can comprise a poly(A), poly(T), poly(C), or poly(G) sequence.

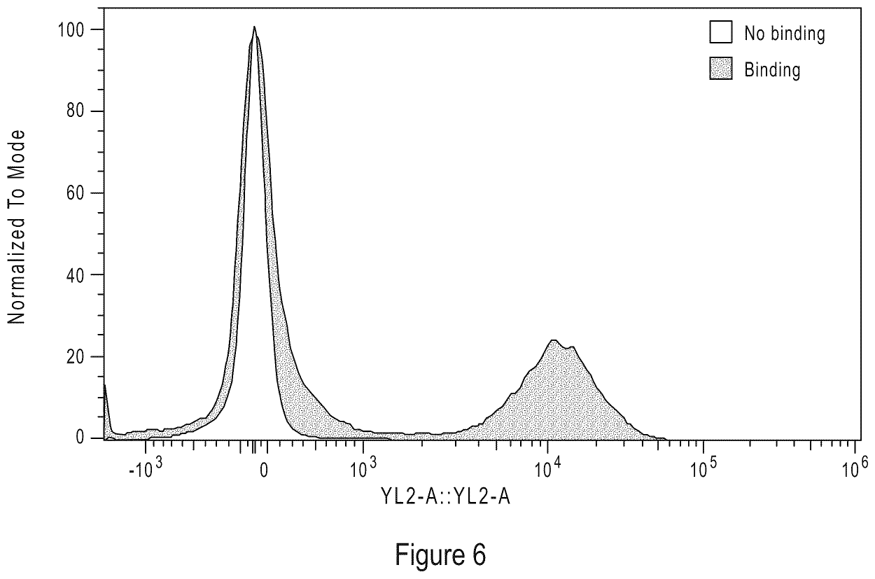

The approach disclosed herein presents numerous heretofore unrealized advantages. For example, a fluorescently labeled nucleic acid nanostructure (also referred to herein as a “genomic fluor”) when bound to sequence-tagged antibodies can be used for validation of commercially available sequence-tagged antibody reagents, e.g. by combining with commercially available sequence-tagged antibody reagents and running a sample by flow cytometry to see if an antibody is binding to cells as expected. This is critically important as it has been observed that commercially available sequence-tagged antibody performance can be changed/degraded by the conjugation process and thus commercially available sequence-tagged antibodies may not bind the target indicated (FIG. 6). This would only be observed after a very expensive sequencing experiment, if at all, given that it in certain instances distinguishing a “true” negative from a “false” negative can be difficult.

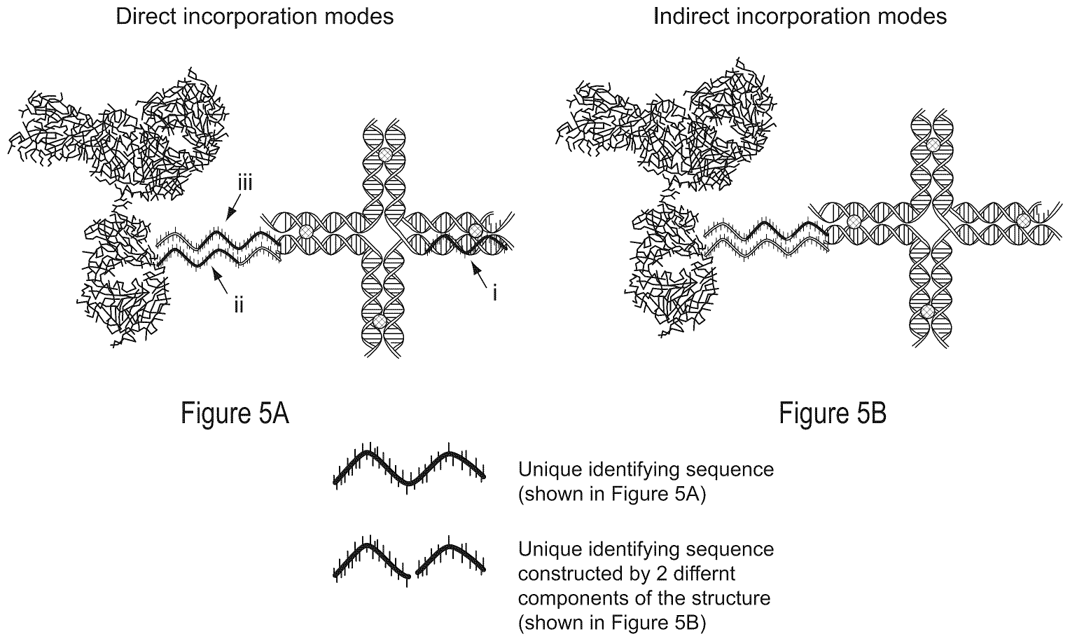

In certain embodiments, by leveraging a nucleic acid nanostructure such as a genomic fluor, one or multiple unique identifying sequences can be incorporated directly into either the “linker” sequence or the nucleic acid nanostructure itself in any location (FIG. 5A shows illustrative example locations of one or multiple unique identifying sequences). In certain embodiments, the unique identifying sequence(s) can be incorporated into construction of the nucleic acid nanostructure itself (FIG. 5A at (i)), or the linker sequence on either end and/or strand of the attachment (FIG. 5A at (ii) and (iii)).

In certain embodiments, a unique identifying sequence could also be at a junction sequence at a point of “assembly” between two oligonucleotides which themselves are part and/or extensions of the nucleic acid nanostructure. For example, described herein is an embodiment in which the unique identifying sequence is constructed “indirectly” with portions contributed by the linker and the nucleic acid nanostructure to construct one unique identifying sequence (FIG. 5B).

In certain embodiments, the positions, construction, and stoichiometric quantity of unique identifying sequences can be tightly controlled, which, as described in greater detail elsewhere herein, has a large impact on the utility of these nucleic acid nanostructures in sequencing-based applications. Further, in certain embodiments, any and all of these modes may be combined.

In certain embodiments, nucleic acid nanostructures can be linked together akin to individual “lego” pieces. At the junctions of these connections between nucleic acid nanostructures, new sequences can be created, leading to a new unique identifying sequence. In addition, this amplification can be used to tune up and down the number of unique identifying sequences as described elsewhere herein in further detail. Additionally, in certain embodiments, nucleic acid nanostructures can be linked to or themselves used as a substrate, such as for an active biological or chemical process to occur (e.g., cleavage of a chemical moiety as a measurement of caspase activity before cell death, or CRISPR editing activity of a specific sequence on or between the nucleic acid nanostructure). Also, in certain embodiments, gene editing modalities can be used to expose compliments or “sticky ends” in order to use specific targeting sequences to construct new unique identifying sequence(s) via the nucleic acid nanostructure itself.

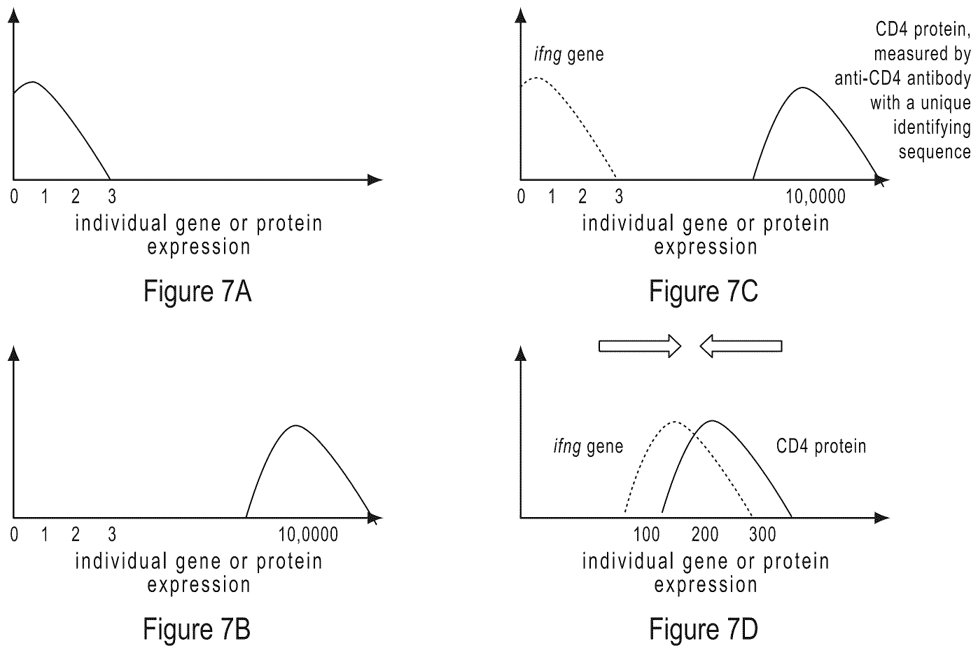

Thus, one significant aspect of the compositions and methods of this disclosure is the ability to reproducibly and quantitatively control the number of unique identifying sequences used in targeting either proteins/antigens/epitopes or genomic material using, for example, an antibody-conjugated nucleic acid nanostructure. Using conjugation chemistry, the inventors have demonstrated the ability to tightly control the degree of labeling (DoL) on an antibody, and in particular, the number of nucleic acid nanostructures attached. Importantly, it has also been observed that commercially available sequence-tagged antibodies may have one or more than one unique identifying sequences. This has an important influence on the quantification of proteins detected by antibodies, as one could interpret expression changes of two-fold simply based on the number of unique identifying sequences, rather than the detection of the underlying proteins. As noted in Table 1 above, the issue of detection at the low end in single-cell gene expression and protein detection through the methods described in Table 2 is very challenging due to the presence of drop-outs. For example, a molecule of RNA or a protein on the surface of a cell that is expressed at 1-3 copies/1-3 proteins. In such a case, the signal of either species could be amplified using the quantitative control of unique identifying sequences and amplification, to bring the fidelity of signal detection above the level of drop-outs. Alternatively, highly expressed proteins, e.g., CD4 proteins, of which about 40,000 molecules are expressed on the surface of a cell, could be “titrated” down using nucleic acid nanostructures that lack unique identifying sequences. In this manner, both low expressed proteins and RNA and high expressed proteins and RNA could be brought into the same dynamic range (see FIG. 7D). This can be done for “genomic” and proteomic/epitope detection, which will also have a dramatic effect on the number of reads necessary and thus the cost of running an experiment. As both RNA and protein are measured in the same experiment, this allows for the measurement “normalization” in sequencing (that is, measuring both RNA and protein together with high fidelity in a narrower dynamic range), while controlling sequencing costs.

In certain embodiments, fluorescent labels and oligo components can be changed independently of one another, which enables fine-tuned control of quantitation and detection in at least two modalities of measurement (e.g., fluorescence-based and sequencing). Furthermore, the control can be used to optimize detection on different detection modalities (which may inherently have different dynamic ranges) and “tune” or titrate signal intensities to account for and discover more about the underlying biology. Additionally, there are several modalities through which the number of nucleic acid nanostructures can be quantitatively and precisely controlled, and by extension, the number of unique identifying sequences, providing for means of amplifying signals for low expression that are non-destructive and do not require complicated workflows.

As noted above, currently in the field there is no way to combine workflows or preserve the optionality following a measurement to make a decision about the ensuing analysis to pursue. The compositions and methods of this disclosure enable new workflows and a whole new way of thinking about an experiment. For example, as fluorescence measurements in many of the workflows discussed herein precede the move to sequencing, the combined measurement modality described enables one to make decisions “in real time” as part of a scientific experiment. For example, one of skill in the art could be sorting cell populations by flow cytometry (FACS) and observe that an additional population is of interest for downstream, deeper, analysis by single cell sequencing. As another example, immunofluorescence imaging could reveal a new section or region of interest for further analysis of spatial gene expression. In both cases, one is able to make these decisions live, during the experiment, and decide which measurements to take for specific populations or tissue regions in a way not previously possible.

Another current limitation is that there is no link to the central canon of biology (cell phenotypes and function). Using single-cell sequencing including commercially available methods (e.g., TotalSeq and droplet-based methods) >100 proteins on individual cells in addition to RNA information can be analyzed, including the whole transcriptome. Concurrently, the ability to achieve >=40 colors (questions per cell) using flow cytometry has been achieved. With these capabilities, one might assume that the field has reached its brahmanic apex, understanding the cellular universe and its ultimate reality. The reality, however, is that rifts between genomic single cell measurements and flow cytometry are due to a complete lack of interoperability.

The embodiments described herein solve these deep problems of linking measurement modalities. Advantageously using compositions and methods of the present disclosure, one reagent can be used to measure the identity of a cell in both fluorescence measurement and in sequencing (in the case of an antibody that is conjugated to a nucleic acid nanostructure that contains the ability to fluoresce and contains at least one unique identifying sequence). Additional advantages include RNA or other -omes can be measured and sorted/imaged through nucleic acid nanostructures that specifically target sequences of interest. According to the current disclosure, the same reagent that is used for upstream enrichment can also be used for downstream analysis. A disadvantage of the current state of the art is that the same clone and specificity of antibody can currently not be used for both upstream and downstream measurements due to blocking of the epitope to which the antibody binds (which will be described in further detail elsewhere herein as epitope blocking).

Another problem with the current state of the art is that data cannot be tied together without complex informatics solutions. This is solved by the compositions and methods provided herein as the same antibody of the same clone and specificity can be used for both enrichment and sequencing because one could use the identity or leverage an “identity barcode” to link the data from the fluorescence measurement to the -omics measurement via NGS. With the one reagent solution disclosed herein, one can use a set of antibodies as tissue “landmarks” to register various measurements in the case of immunofluorescence imaging preceding gene expression measurement. As many of the latter measure regions of gene expression rather than gene expression within individual cells, this enables the mapping of gene expression to individual cells.

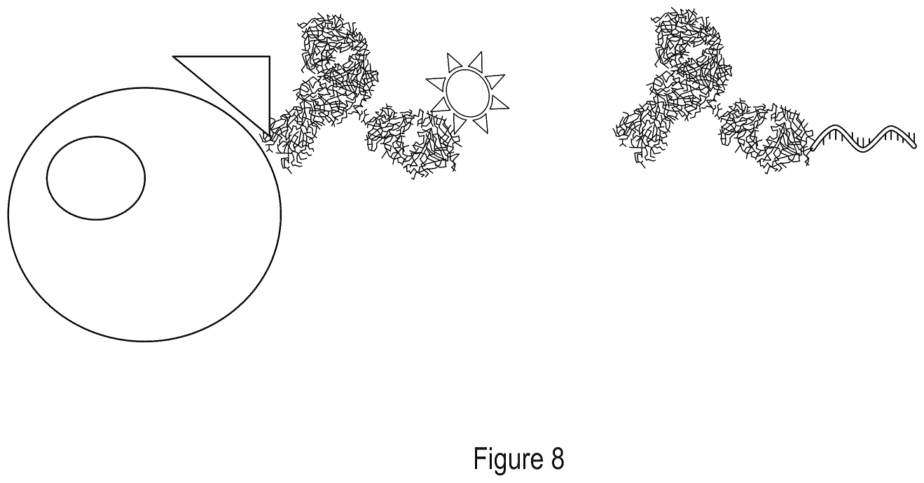

Another problem with the current state of the art that is also a cost driver, validation nightmare, and scientifically limiting aspect of the current technology is that different antibodies must be used for the enrichment/imaging step and downstream analysis by sequencing (FIG. 8), e.g., epitope/antigen blocking and validation is impossible in current paradigms, and 2× antibodies are purchased for each target. This is because after staining with fluorescent dye conjugated antibodies, the epitope targeted by the antibody is now blocked and cannot be stained again. This is very limiting, as antibodies have different performance (affinity) based on their clones (and epitopes recognized) and thus a scientist may have to use a worse performing antibody to measure a protein in both fluorescent and sequencing modalities. Additionally, for an individual specificity, there may only be one available antibody, thus one would have to choose to measure this protein in either the fluorescence or sequencing measurement. One of ordinary skill in the art would also understand that some antibodies also block other nearby epitopes on proteins due to their size.

In a standard experiment, 10 antibodies would be used for sorting and an additional 100 sequence-tagged antibodies would be measured simultaneously with whole transcriptome analysis (WTA). Thus, the 10 antibody clones/specificities used in the upstream fluorescence measurements cannot be used for the downstream sequence-tagged antibodies. Additionally, in this current experiment 110 antibodies must be purchased, whereas in embodiments of the present disclosure, 100 antibodies could be purchased and any of them could be measured using fluorescent or genomic modalities. As many hundreds of proteins can be measured in one experiment, this also enables large-scale validation of antibodies before sequencing or downstream genomic-reliant measurement. It has been observed that the performance (affinity) of some commercially available antibodies is negatively affected by the chemistry used to conjugate an oligonucleotide to said antibody. In an experiment with many hundreds of antibodies, this raises several issues, e.g., if a protein measurement comes up zero, is that due to low expression (and drop-outs) or because the antibody was not functioning properly? It also raises the question of how does one validate a sequence-tagged antibody? With the methods and compositions disclosed herein, one can validate current sequence-tagged antibodies by fluorescence measurement or immediate validation is provided by the combined reagent. That is, certain embodiments provide for a method of validating a sequence-tagged antibody wherein the method comprises contacting a genomic fluor comprising a nucleic acid linker with a sequence-tagged antibody and running a sample by flow cytometry to evaluate the antibody's binding to its target.

Provided herein are methods through which fluorescence measurements of biomolecules can be combined on the same sample with a genomic read-out. Using the compositions and methods disclosed herein, new multimodal workflows are possible in both single-cell suspension and imaging applications, for example, as illustrated in Table 4.

TABLE 4
How the disclosure addresses the challenges of current single-cell analyses in solution.

| | Solutions presented/advantages |
|---|---|
| Challenges | of the disclosure |
| All are reliant on underlying sequencing cost. | can control sequencing cost by making sorting |
| | decisions in real time and validating sequence- |
| | tagged antibodies in a way not previously possible |
| | (in “real time”) |
| can be combined together into multi-modal | staining for FACS-driven cell enrichment can be |
| experiments, but sequencing cost is very | done all in one step as described herein |
| prohibitive so cell enrichment, e.g. by magnetic | |
| bead separation and/or flow cytometry activated | |
| cell sorting (FACS), is critical (cost), as described | |
| above | |
| cannot currently use the same reagent for this and | fundamentally solved herein, a single reagent can |
| fluorescence measurement, as the antibody blocks | be used in both workflows |
| the binding on that epitope, limiting combined | |
| workflows | |
| technical difficulties | N/A these are system (hardware) specific |
| C1: doublets | |
| DropSeq: technically complex to establish | |
| in a lab | |
| ddSeq: Poisson capture rate | |

In certain embodiments, the compositions (e.g., reagents) of this description can be used in one or in combination of any methods listed in Table 1 and Table 2 and address the significant cost and detection issues as aforementioned. It will be understood to one of ordinary skill in the art that the compositions and methods described herein can be used in bulk tissue measurement (e.g. bulk RNA-Seq), single-cell measurement (e.g., through droplet based techniques or others), as well as imaging, and with methods of using a combination of fluorescence label and genomic tag/unique identifying sequence. Such signals can be detected by a large range of detection modalities including imaging, flow cytometry, microscopy, etc. and for the latter, NGS, PCR, RT-PCR, etc. Furthermore, provided for herein are validation and real-time decision-making capabilities to a scientific workflow incorporating one or many of the modalities examining single-cell suspensions. Embodiments of the present disclosure are understood to cover methods using both 3′ and 5′ approaches for single cell sequencing. While 3′ sequencing is predominantly used e.g., for whole transcriptome analysis based on the relative ease of capturing the poly(T) tail of messenger RNA, 5′ sequencing enables analysis of T and B cell immune receptors, often in combination with other measurements. In certain embodiments, this can be used in targeted RNA sequencing as well, in which a handful of genes (e.g. 100-1000) is chosen for sequencing. The method of leveraging the embodiments provided herein above across platforms and workflows to enable decision making is a key method innovation, as is the ability to link data on the same set of proteins/cells in the downstream analysis.

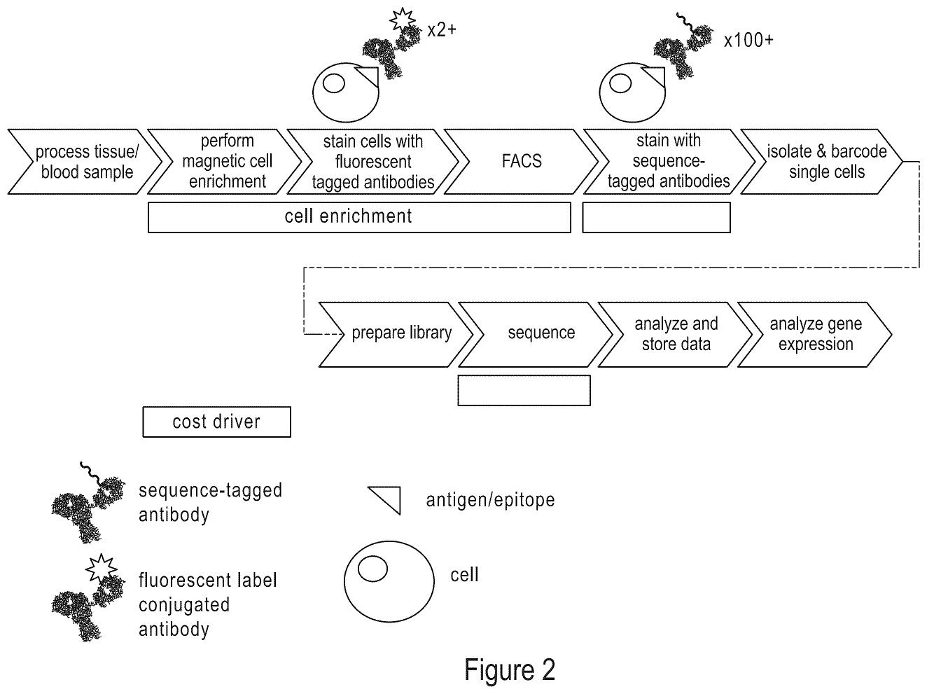

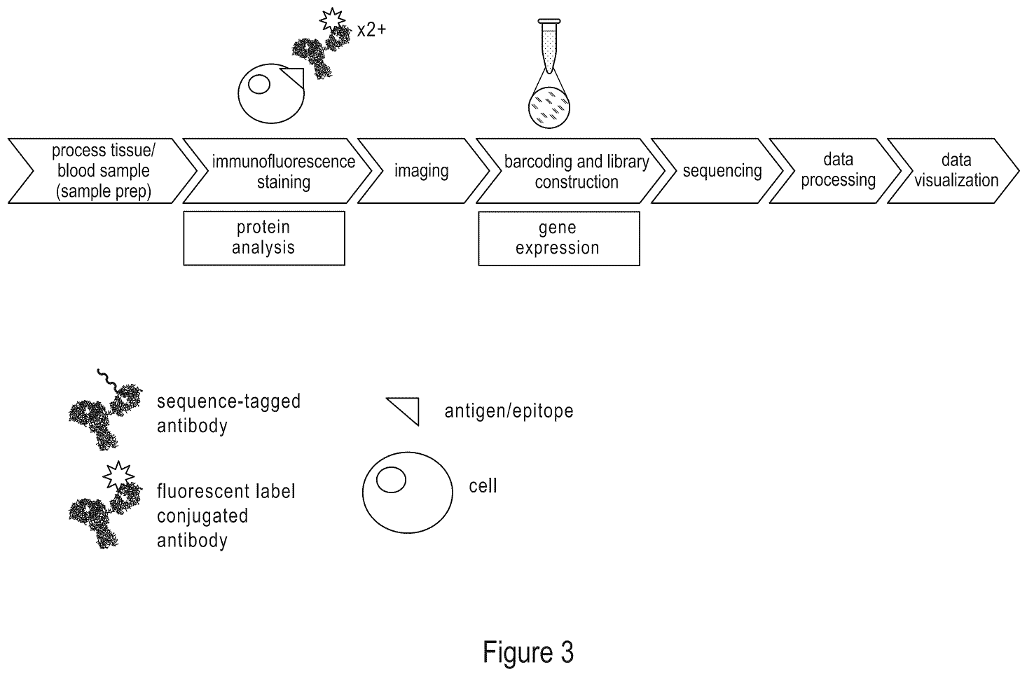

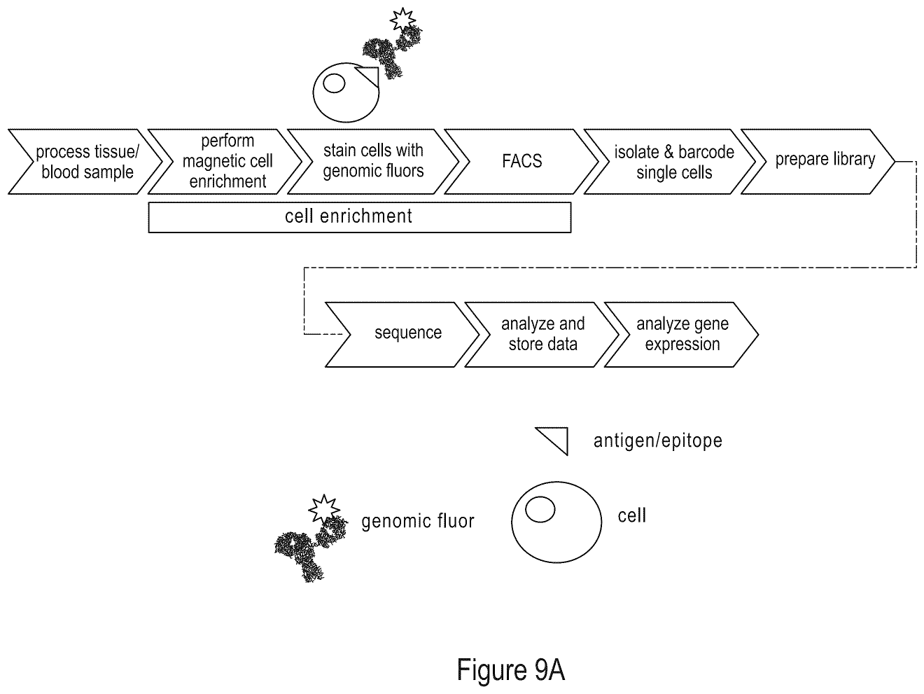

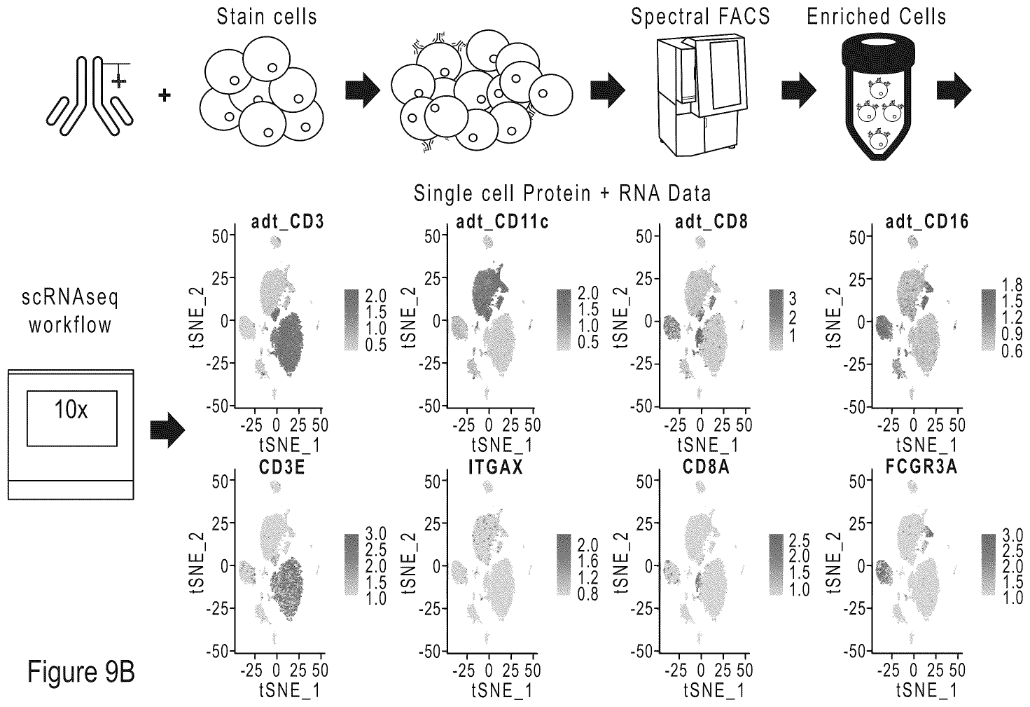

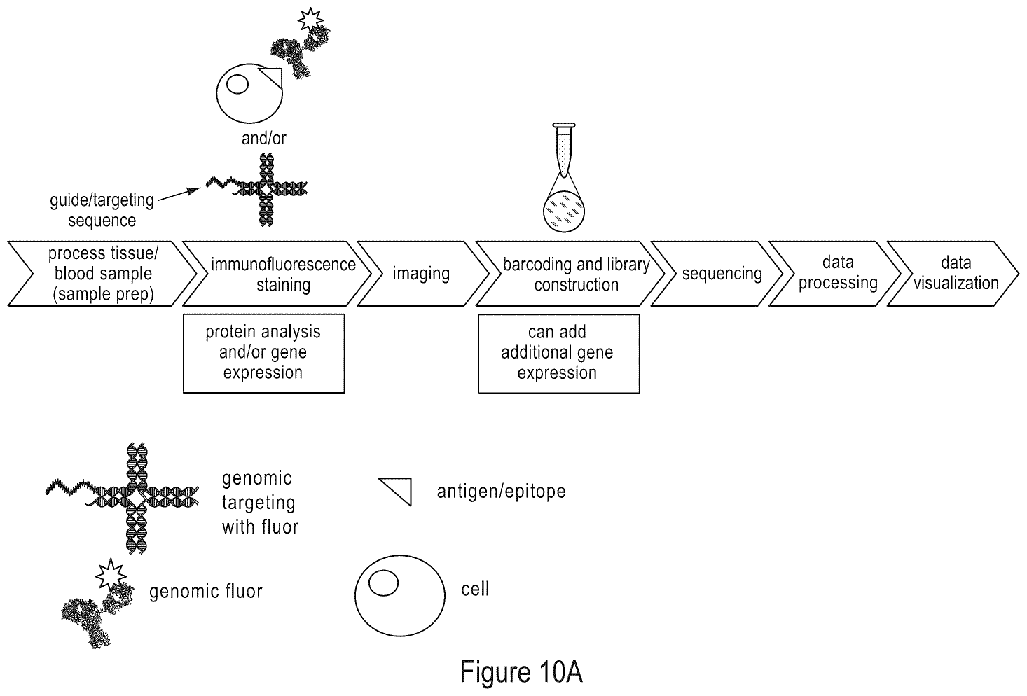

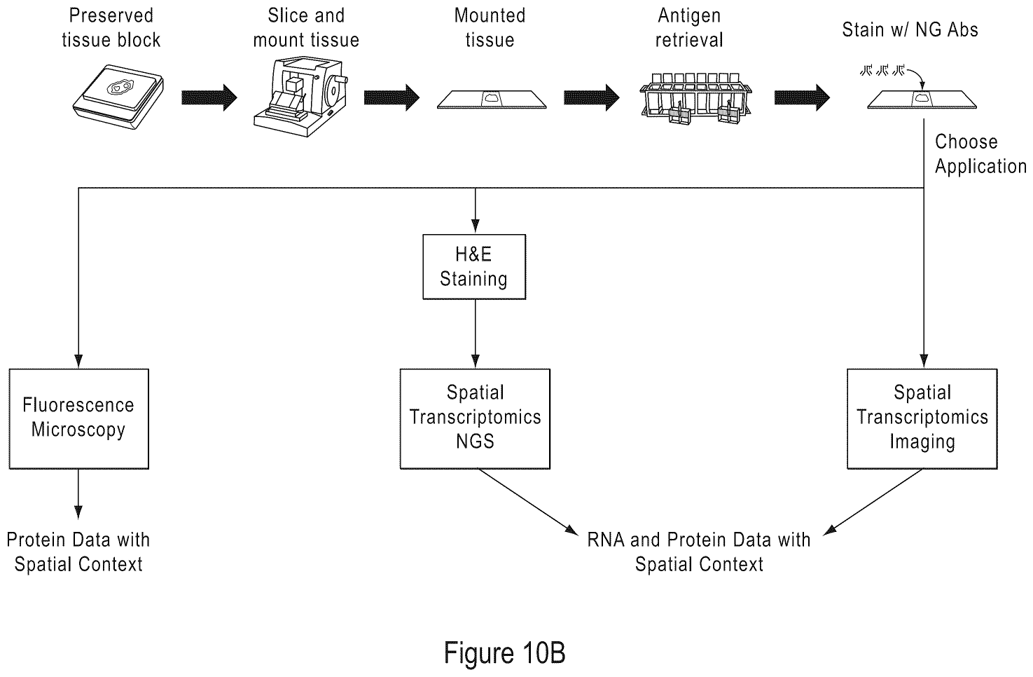

Based on the current single-cell sequencing workflow with sequence-tagged antibodies (FIG. 2), which suffers from the various problems herein described, new “unified” workflows and methods as described in FIG. 9A through FIG. 10B are disclosed herein. This one-step labeling is a key methods innovation with broad applicability across the platforms described herein. The methods of the disclosure described herein also allow for multi-step labeling, with the critical difference that the same reagent can be used for reagent and enrichment in a way never before possible.

TABLE 5
Further advantages of the embodiments of the disclosure.

| | Solutions presented/advantages |
|---|---|
| Approach | of this embodiment |
| Traditional IF | increase and fully utilize |
| | multiplex imaging with |
| | spectrally clean fluors |
| | remove reliance on secondary |
| | antibodies with |
| | bright signals |
| Mass Spec | spectrally clean > mass clean |
| (IMC ™ or MIBI ™) | and no tissue |
| | destruction |
| Ultivue's | provide higher plex panels, with all fluors |
| InSituPlex ® | no amplification needed |
| Sequential | sequential staining without |
| (MultiOmyx ™, Opal ™) | harsh chemistry |
| | complicated workflows |
| | heat inactivation |
| CODEX ® | simpler sequential staining, one reagent |

The embodiments of this disclosure have broad applicability to imaging alone and in combination with genomic measurements; for example, the use of the genomic fluor and its various embodiments in the various methods described in Table 3 and Table 5. In certain embodiments for single-cell suspension methods, workflows may combine one or more of those techniques as well. Representative applicable imaging methods including single photon microscopy, intravital microscopy, super resolution microscopy, whole tissue imaging, and traditional fluorescence microscopy (IF-IC (immunocytochemistry), IF-F (frozen), and mIHC (multiplexed immunohistochemistry). Further, certain embodiments can be used across a broad range of tissue preparations (e.g., cultured cell lines; primary cells; frozen tissue; and formalin-fixed, paraffin-embedded (FFPE) tissue).

Certain embodiments described herein can increase the resolution of extant platforms by providing tissue landmarks. In this way, one can overcome the problem where transcriptome data is only analyzed for tissue regions rather than individual cells.

Additional challenges presented by attempting to assay both protein and RNA/genomic material in one workflow include that the steps of protein measurement and genomic measurement are separate steps. While the latter can easily achieve single-cell resolution, in some embodiments it only captures regions of gene expression. In certain embodiments of the present disclosure however, a genomic fluor for example, can be used to target protein antigens (or other epitopes) and alternatively be constructed in such a way that it targets genomic material e.g., using a guide sequence, measurements of both protein and genomic materials could be combined in new ways (e.g. FISH and IF). It has been observed by the inventors that the genomic fluors described herein also display very bright staining, thus potentially obviating the problematic use of secondary antibodies in imaging applications. In addition, it has been demonstrated that genomic fluors have access to both the cytosol and nucleus, enabling a very broad range of measurements. In certain embodiments, a genomic fluor also enables “optimize once” use of new workflows, as >90% of the mass of, for example a PHITON genomic fluor, is made up of DNA. Thus, FIGS. 10A and 10B present examples of a new workflow for combining immunofluorescence imaging and gene expression. In certain embodiments, amplification can be added but is not required for imaging.

### Multimodal Methods

Provided for herein is a method for combining cell enrichment with genomic analysis. Provided for herein is a method for combining cell sorting with genomic analysis. Provided for herein is a method for combining immunofluorescent cell labeling with genomic analysis. Certain embodiments provide for a combination of cell enrichment, cell sorting, and/or immunofluorescent cell labeling with genomic analysis. While not limited by any particular cell enrichment or cell sorting method, in certain embodiments, the cell enrichment and/or cell sorting is performed by flow cytometry/FACS. While not limited by any particular type of fluorescent labeling, in certain embodiments the fluorescent labeling comprises visualization and/or quantitation such as with single- or multi-photon microscopy, intravital microscopy, super resolution microscopy, whole tissue imaging, traditional fluorescence microscopy (IF-IC (immunocytochemistry)), IF-F (frozen), and/or mIHC (multiplexed immunohistochemistry). While not limited by any particular genomic analysis method, in certain embodiments the genomic analysis comprises Sanger sequencing, next generation sequencing (NGS), long-read sequencing, in situ sequencing, polymerase chain reaction (PCR), and/or reverse transcription polymerase chain reaction (RT-PCR). The method is enabled by the use of a “fluorescent-labeled sequence-tagged specificity determining molecule conjugate” (“conjugate”), wherein one or more components of the conjugate are used for the cell enrichment, cell sorting, and/or immunofluorescent cell labeling protocol(s) and one or more components of the same conjugate are utilized in the genomic analysis protocol(s). In certain embodiments, the method uses a fluorescent-labeled sequence-tagged specificity determining molecule conjugate to identify a single cell by both fluorescent measurement and sequencing. In certain embodiments, the use of a fluorescent-labeled sequence-tagged specificity determining molecule conjugate allows data from cell enrichment, cell sorting, and/or immunofluorescent cell labeling to be linked to data from genomic analysis. And, in certain embodiments, the method is applied to a single-cell suspension, bulk tissue measurement, or an imaging application.

In certain embodiments, the method comprises (a) performing cell enrichment, cell sorting, and/or immunofluorescent cell labeling on a cell and/or sample of cells and also (b) performing genomic analysis on the same cell and/or sample of cells, using the fluorescent-labeled sequence-tagged specificity determining molecule conjugate.

While the methods are not limited by the order in which the different protocols occur/modalities are measured, in certain embodiments, the genomic analysis occurs after the cell enrichment, cell sorting, and/or immunofluorescent cell labeling. As discussed above, compositions and methods of this disclosure enable one to make decisions, even “in real time,” as part of a scientific experiment which types of protocols, analysis, measurements, modalities, etc., to perform and combine in ways not previously possible. In certain embodiments, the choice of cell enrichment, cell sorting, and/or immunofluorescent cell labeling method is not limiting on the choice of genomic analysis method, whether upstream or downstream. Further, in certain embodiments, the choice of genomic analysis method can be based on the results of the cell enrichment, cell sorting, and/or immunofluorescent cell labeling.

In certain embodiments, the fluorescent-labeled sequence-tagged specificity determining molecule conjugate can be made to specifically identify and/or bind a target molecule via its specificity determining molecule component. In certain embodiments, the specificity determining molecule comprises a protein, enzyme, carbohydrate, nucleic acid, receptor, receptor ligand, and/or substrate that enable the assaying of different -omes (e.g, transcriptome, epigenome, genome, and proteome). In certain embodiments, the specificity determining molecule is a binding molecule such as an antibody or an antigen-binding fragment, variant, or derivative thereof. In certain embodiments, the binding molecule is a peptide, recombinant, natural, or engineered receptor/ligand protein, aptamers, tetramers (folded MHC proteins with peptides used for detecting T cell receptors), non-antibody proteins or antibody mimetics, e.g., affilins, affimers, affitins, alphabodies, avimers, fynomers, Kunitz domain peptides, nanoCLAMPS, Designed Ankyrin Repeat Proteins (DARPins), monobodies, nanobodies, anticalins, affibodies, and/or SOMAmers. In certain embodiments, the binding molecule is a receptor ligand. In certain embodiments, the specificity determining molecule is a nucleic acid such as comprising a nucleic acid sequence that can target another nucleic acid sequence. For purposes of this disclosure, a fluorescent-labeled nucleic acid nanostructure which incorporates a target targeting sequence is considered to comprise both a fluorescent label component and a specificity determining molecule component.

In certain embodiments, the fluorescent-labeled sequence-tagged specificity determining molecule conjugate can comprise as its fluorescent label one or more fluorescently labeled nucleic acid nanostructure referred to herein as a “genomic fluor.” In certain embodiments, a nucleic acid nanostructure and/or genomic fluor comprises one or more naturally occurring or synthetic nucleic acid strands. In certain embodiments, a genomic fluor comprises multiple distinct nucleic acid nanostructures (e.g., nucleic acid nanostructure units) linked together. By controlling the number of fluorescently labeled nucleic acid nanostructures that are part of the genomic fluor of a fluorescent-labeled sequence-tagged specificity determining molecule conjugate and/or the number of fluorescent moieties per nucleic acid nanostructure, one can precisely control, determine, and/or manipulate the fluorescent signal of the conjugate. A fluorescently labeled nucleic acid nanostructure can attribute its fluorescence to the incorporation of fluorophores/fluorescent moieties (an example of which is a PHITON) or can incorporate fluorescent nucleic acids. In certain embodiments, a fluorescently labeled nucleic acid nanostructure can also comprise one or more unique identifying sequences. Such a dual fluorescent label/unique identifying sequence containing moiety of the fluorescent-labeled sequence-tagged specificity determining molecule conjugate is for purposes of this disclosure a “multimodal label.” Further, in certain embodiments, the fluorescent-labeled sequence-tagged specificity determining molecule conjugate can also comprise one or more dark nucleic acid nanostructures, i.e., with no fluorescent label, such as containing no label whatsoever or comprising one or more unique identifying sequences but no fluorescent label. By controlling the number of unique identifying sequences incorporated into the nucleic acid nanostructures of a fluorescent-labeled sequence-tagged specificity determining molecule conjugate, as well as any unique identifying sequences attached to the specificity determining molecule and/or incorporated into any nucleic acid linker sequences, the sequencing signal of the conjugate can be controlled, determined, and/or manipulated. Further, in certain embodiments, the fluorescent-labeled sequence-tagged specificity determining molecule conjugate can also comprise one or more nucleic acid nanostructures with a quenching molecule (“quencher”).

In certain embodiments, the fluorescent-labeled sequence-tagged specificity determining molecule conjugate comprises a unique identifying sequence that enables, for example, use in genomic analysis. In certain embodiments, the unique identifying sequence is located adjacent to or in close enough proximity to a nucleic acid primer sequence for replication, amplification, etc., referred to as “associated with,” the unique identifying sequence. In certain embodiments, the sequence-tagged specificity determining molecule conjugate comprises a plurality of unique identifying sequences, for example between any of about 2, 3, 4, 5, or 6 to any of about 4, 5, 6, 8, 10, or 12 unique identifying sequences. For example, 2, 3, 4, 5, or 6 unique identifying sequences. In certain embodiments, the unique identifying sequence is formed from a combination of sequences of two or more separate components, for example, a combination of sequences from separate nucleic acid nanostructures. In certain embodiments, sequences from one or more components are combined with sequences from one or more other components, to create a plurality of unique identifying sequences.

In certain embodiments, a unique identifying sequence can be part of and/or attached to the specificity determining molecule, for example wherein the specificity determining molecule is an antibody and the antibody is a sequence-tagged antibody. In certain embodiments, a unique identifying sequence can be incorporated into the nucleic acid sequence of a nucleic acid nanostructure of the conjugate including, but not limited to, the nucleic acid sequence of a genomic fluor. In certain embodiments, a unique identifying sequence can be incorporated into a linker or linkers used to conjugate one or more components of the conjugate together, such as linking a specificity determining molecule to a fluorescent label, for example linking an antibody to a genomic fluor, or linking multiple nucleic acid nanostructures of the conjugate together. Certain embodiments utilize a combination of such locations. In certain embodiments, a linker also comprises a poly(A), poly(T), poly(C), and/or poly(G) sequence.

The measurements described herein are performed using hardware with constraints on their inherent dynamic range, be that the detection of light e.g., in immunofluorescence imaging or flow cytometry, or the number of RNA species by next-generation sequencing. Often, the biological dynamic range exceeds that of instrumentation—for example attempting to measure a protein that is expressed at very high amounts (e.g. actin) which would be off-scale in the positive direction and a very low expressed protein (e.g. a transcription factor) which would be off-scale in the direction of zero. Using the multi-modal label described herein, both can quantitatively and controllably be brought into the measurement dynamic range of an instrument, in effect, “normalizing” the biological signals so they can be measured accurately using existing instruments. In certain embodiments, the degree-of-labeling (DoL) (also referred to in the art as dye to protein (D:P) or fluorophore to protein (F:P)) of the specificity determining molecule is controlled stoichiometrically. This can be achieved, for example, via the availability of the nucleic acid to be attached. In certain embodiments, the DoL of the specificity determining molecule is used to increase (tune up), decrease (tune down), and/or otherwise control the signal detection. In certain embodiments, the DoL is between any of 1, 2, 3, 4, 5, 6, 7, 8, 9, 10, 11, 15, 20, or 25 and any of 2, 3, 4, 5, 6, 7, 8, 9, 10, 11, 15, 20, 25, 30, 40, or 50. In certain embodiments, the DoL is between any of 1, 2, 3, 4, 5, 6, 7, 8, 9, or 10 and any of 2, 3, 4, 5, 6, 7, 8, 9, 10, or 11. In certain embodiments, the DoL is greater than 25, 50, or 100. Further, in certain embodiments, the fluorescent label is a genomic fluor and the number of fluorophores incorporated into the genomic fluor and/or number of nucleic acid nanostructure components comprising the genomic fluor is used to increase, decrease, or otherwise control the signal detection. In certain embodiments, the sequencing signal of a targeted component is titrated downward by the use of a specificity determining molecule lacking a unique identifying sequence. In certain embodiments, the above techniques or a combination of such techniques can be used to bring the signal of a lowly expressed targeted component and a highly expressed targeted component into the same dynamic range.

### Fluorescent-labeled Sequence-tagged Specificity Determining Molecule Conjugates

As described above, the multimodal methods of the present disclosure are enabled by the use of a fluorescent-labeled sequence-tagged specificity determining molecule conjugate. Thus, the present disclosure provides for a fluorescent-labeled sequence-tagged specificity determining molecule conjugate suitable for use in any of the methods of this disclosure. And the methods of this disclosure can be performed using any of the fluorescent-labeled sequence-tagged specificity determining molecule conjugates disclosed herein.

In certain embodiments, the fluorescent-labeled sequence-tagged specificity determining molecule conjugate can be made to specifically identify and/or bind a target molecule via its specificity determining molecule component. In certain embodiments, the specificity determining molecule comprises a protein, enzyme, carbohydrate, nucleic acid, receptor, receptor ligand, and/or substrate that enable the assaying of different -omes (e.g, transcriptome, epigenome, genome, and proteome). In certain embodiments, the specificity determining molecule is a binding molecule such as an antibody or an antigen-binding fragment, variant, or derivative thereof. In certain embodiments, the binding molecule is a peptide, recombinant, natural, or engineered receptor/ligand protein, aptamers, tetramers (folded MHC proteins with peptides used for detecting T cell receptors), non-antibody proteins or antibody mimetics, e.g., affilins, affimers, affitins, alphabodies, avimers, fynomers, Kunitz domain peptides, nanoCLAMPS, Designed Ankyrin Repeat Proteins (DARPins), monobodies, nanobodies, anticalins, affibodies, and/or SOMAmers. In certain embodiments, the binding molecule is a receptor ligand. In certain embodiments, the specificity determining molecule is a nucleic acid such as comprising a nucleic acid sequence that can target another nucleic acid sequence. For purposes of this disclosure, a fluorescent-labeled nucleic acid nanostructure which incorporates a target targeting sequence is considered to comprise both a fluorescent label component and a specificity determining molecule component.

In certain embodiments, the fluorescent-labeled sequence-tagged specificity determining molecule conjugate can comprise as its fluorescent label one or more fluorescently labeled nucleic acid nanostructure referred to herein as a “genomic fluor.” In certain embodiments, a nucleic acid nanostructure and/or genomic fluor comprises one or more naturally occurring or synthetic nucleic acid strands. In certain embodiments, a genomic fluor comprises multiple distinct nucleic acid nanostructures (e.g., nucleic acid nanostructure units) linked together. By controlling the number of fluorescently labeled nucleic acid nanostructures that are part of the genomic fluor of a fluorescent-labeled sequence-tagged specificity determining molecule conjugate and/or the number of fluorescent moieties per nucleic acid nanostructure, one can precisely control, determine, and/or manipulate the fluorescent signal of the conjugate. A fluorescently labeled nucleic acid nanostructure can attribute its fluorescence to the incorporation of fluorophores/fluorescent moieties (an example of which is a PHITON) or can incorporate fluorescent nucleic acids. In certain embodiments, a fluorescently labeled nucleic acid nanostructure can also comprise one or more unique identifying sequences. Such a dual fluorescent label/unique identifying sequence containing moiety of the fluorescent-labeled sequence-tagged specificity determining molecule conjugate is for purposes of this disclosure a “multimodal label.” Further, in certain embodiments, the fluorescent-labeled sequence-tagged specificity determining molecule conjugate can also comprise one or more dark nucleic acid nanostructures, i.e., with no fluorescent label, such as containing no label whatsoever or comprising one or more unique identifying sequences but no fluorescent label. By controlling the number of unique identifying sequences incorporated into the nucleic acid nanostructures of a fluorescent-labeled sequence-tagged specificity determining molecule conjugate, as well as any unique identifying sequences attached to the specificity determining molecule and/or incorporated into any nucleic acid linker sequences, the sequencing signal of the conjugate can be controlled, determined, and/or manipulated. Further, in certain embodiments, the fluorescent-labeled sequence-tagged specificity determining molecule conjugate can also comprise one or more nucleic acid nanostructures with a quenching molecule (“quencher”).

A “unique identifying sequence” is an oligonucleotide sequence that can be used to distinguish between one or multiple species, for example, one to which a complementary primer sequence can bind to for downstream amplification (such as by PCR), long-read sequencing, next generation sequencing (NGS), in situ sequencing or alternatively, that can be probed using a complementary sequence, e.g., through fluorescent in situ hybridization (FISH). In certain embodiments, a double-stranded segment of a nucleic acid linker comprises a unique identifying sequence. In certain embodiments, the unique identifying sequence can be used for nucleic acid amplification such as by PCR. In certain embodiments, the unique identifying sequence can be used for next-generation sequencing (NGS). In certain embodiments, a nucleic acid linker comprises a sequence enabling it to be filtered out in downstream sequencing applications. For example, wherein the sequence is distinguishable from other nucleotide sequences in sequencing through the use of a unique sequence, e.g., one to which a complementary primer sequence can bind, enabling filtering of all linker-tagged species to be excluded from downstream analysis. In certain embodiments, the nucleic acid linker comprises a sequence for specific binding by a third biomolecule and/or for targeted gene editing through enzymatic cleavage (e.g., CRISPR, Zinc-finger nucleases, restriction enzymes). For example, wherein the sequence is designed to enable targeting through a CRISPR gRNA and this targeting is cleaved by CRISPR, or for example, a target site for the DNA-binding domain of a Zinc-finger nuclease, or alternatively, a sequence that is specifically targeted for cleavage by a restriction enzyme, e.g., EcoRI endonuclease, which cleaves the DNA sequence GAATTC. In certain embodiments, the nucleic acid linker comprises one or more unique sequences enabling enzymatic or binding activity. In certain embodiments, a unique sequence enabling enzymatic or binding activity is present in the double-stranded segment.

In certain embodiments, the fluorescent-labeled sequence-tagged specificity determining molecule conjugate comprises a unique identifying sequence that enables, for example, use in genomic analysis. In certain embodiments, the unique identifying sequence is located adjacent to or in close enough proximity to a nucleic acid primer sequence for replication, amplification, etc., referred to as “associated with,” the unique identifying sequence. In certain embodiments, the sequence-tagged specificity determining molecule conjugate comprises a plurality of unique identifying sequences, for example between any of about 2, 3, 4, 5, or 6 to any of about 4, 5, 6, 8, 10, or 12 unique identifying sequences. For example, 2, 3, 4, 5, or 6 unique identifying sequences. In certain embodiments, the unique identifying sequence is formed from a combination of sequences of two or more separate components, for example, a combination of sequences from separate nucleic acid nanostructures. In certain embodiments, sequences from one or more components are combined with sequences from one or more other components, to create a plurality of unique identifying sequences.

In certain embodiments, a unique identifying sequence can be part of and/or attached to the specificity determining molecule, for example wherein the specificity determining molecule is an antibody and the antibody is a sequence-tagged antibody. In certain embodiments, a unique identifying sequence can be incorporated into the nucleic acid sequence of a nucleic acid nanostructure of the conjugate including, but not limited to, the nucleic acid sequence of a genomic fluor. In certain embodiments, a unique identifying sequence can be incorporated into a linker or linkers used to conjugate one or more components of the conjugate together, such as linking a specificity determining molecule to a fluorescent label, for example linking an antibody to a genomic fluor, or linking multiple nucleic acid nanostructures of the conjugate together. Certain embodiments utilize a combination of such locations. In certain embodiments, a linker also comprises a poly(A), poly(T), poly(C), and/or poly(G) sequence. In certain embodiments, the poly(A), poly(T), poly(C), or poly(G) sequence is at least three, four, five, or six nucleotides in length.

In certain embodiments, a specificity determining molecule, e.g., a sequence-tagged specificity determining molecule, is linked to the fluorescent label by a nucleic acid linker. In certain embodiments, the nucleic acid linker is single-stranded, at least partially double-stranded, or entirely double-stranded. In certain embodiments, the nucleic acid linker is a hybridized at least partially double-stranded nucleic acid, the specificity determining molecule is covalently attached to one strand of the linker, the fluorescent label is covalently attached to the opposite strand of the linker. However, in certain embodiments, the specificity determining molecule and the fluorescent label are not covalently attached but instead linked via the hybridization of their respective linker strands.

The nucleic acid linker can be of any length but certain considerations can be taken into account. For example, an extremely short linker may bring conjugate components into too close of contact, resulting in steric hindrance or other interference. On the other hand, a very long linker may be more difficult to produce or may not keep the components within an optimal distance. In certain embodiments, the nucleic acid linker is at least about 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. In certain embodiments, the nucleic acid linker is from any of about 10, 11, 12, 13, 14, 15, 16, 17, 18, 19, 20, 25, 30, 35, 40, 50, or 60 nucleotides in length to any of about 11, 12, 13, 14, 15, 16, 17, 18, 19, 20, 25, 30, 35, 40, 50, 60, or 75 nucleotides in length. In certain embodiments, the nucleic acid linker is from any of about 10, 15, 20, 25, 30, 35, 40, or 50 nucleotides in length to any of about 15, 20, 25, 30, 35, 40, 50, or 75 nucleotides in length. In certain embodiments, the nucleic acid linker is from any of about 15, 20, 25, 30, or 35 nucleotides in length to any of about 20, 25, 30, 35, or 40 nucleotides in length. The nucleic acid linker can include both single-stranded and double-stranded segments. In certain embodiments, the double-stranded segment of the nucleic acid linker is at least about 10, 15, 20, 25, 30, 35, 40, 45, 50, 55, 60, 65, 70, 75, 80, 85, 90, 95 or 100 nucleotides in length. In certain embodiments, the double-stranded segment of the nucleic acid linker is any of about 10, 15, 20, 25, 30, 35, 40, 45, 50, 55, 60, 65, 70 or 75 nucleotides in length to any of about 15, 20, 25, 30, 35, 40, 45, 50, 55, 60, 65, 70, 75, 80, 85, 90, 95 or 100 nucleotides in length. One of ordinary skill in the art will recognize that whereas double-stranded nucleic acids are generally thought to be made of annealed sequences of complementary base pairs, not all the pairing in a double-stranded nucleic acid segment need be complementary. There is some tolerance for two strands of nucleic acids comprising complementary bases to anneal to form a double-stranded nucleic acid incorporating some non-complementary base paring. Also, degenerate (universal) bases such as deoxyinosine exist that can pair with numerous bases. In certain embodiments, the double-stranded segment of the nucleic acid linker comprises at least about 10, 15, 20, 25, 30, 35, 40, 45, 50, 55, 60, 65, 70, or 75 complementary base pairs, even if the double-stranded segment is not entirely composed of complementary base pairs. In certain embodiments, the double-stranded segment of the nucleic acid linker comprises from any of about 10, 15, 20, 25, 30, 35, 40, 50, or 60 complementary base pairs to any of about 15, 20, 25, 30, 35, 40, 50, 60, or 75 complementary base pairs, even if the double-stranded segment is not entirely composed of complementary base pairs. In certain embodiments, at least 85%, 90%, 95%, or 98% of the double-stranded segment of the nucleic acid linker is complementary base paired. In certain embodiments, the double-stranded segment of the nucleic acid linker has no more than 1, 2, 3, 4, 5, 6, 7, 8, 9, 10, or 11 mismatched base pairs. In certain embodiments, however, 100% of the double-stranded segment is complementary base paired. In certain embodiments, the double-stranded segment comprises at least about 7, 8, 9, 10, 11, 12, 13, 14, 15, 16, 17, 18, 19, 20, 25, 30, 40, 50, 60, or 75 consecutive complementary base pairs or from any of about 7, 8, 9, 10, 15, 20, 25, 30, 35, 40, 50, or 60 consecutive complementary base pairs to any of about 8, 9, 10, 15, 20, 25, 30, 35, 40, 50, 60, or 75 consecutive complementary base pairs.

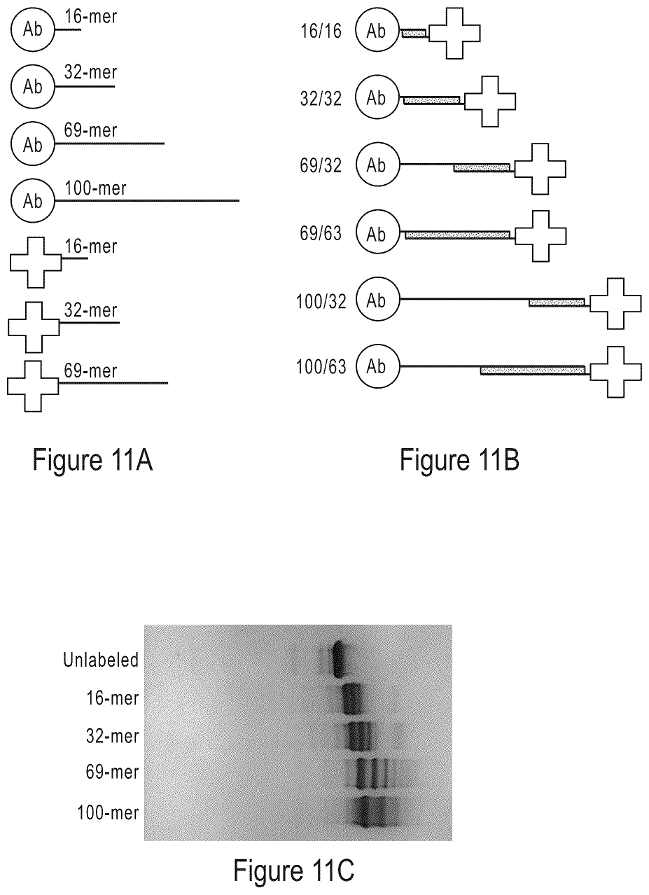

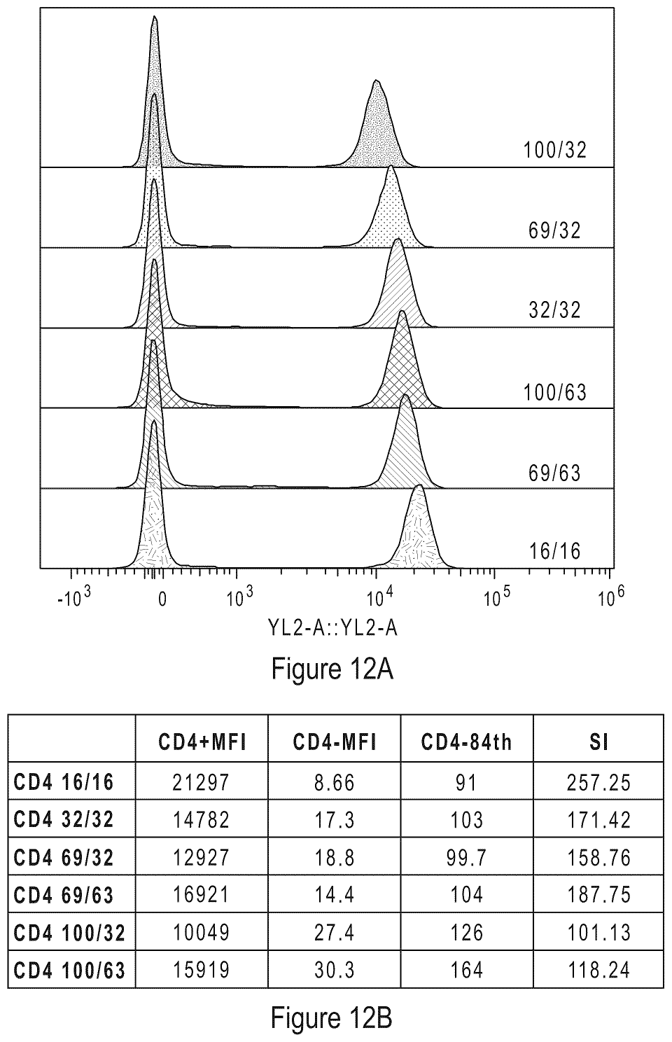

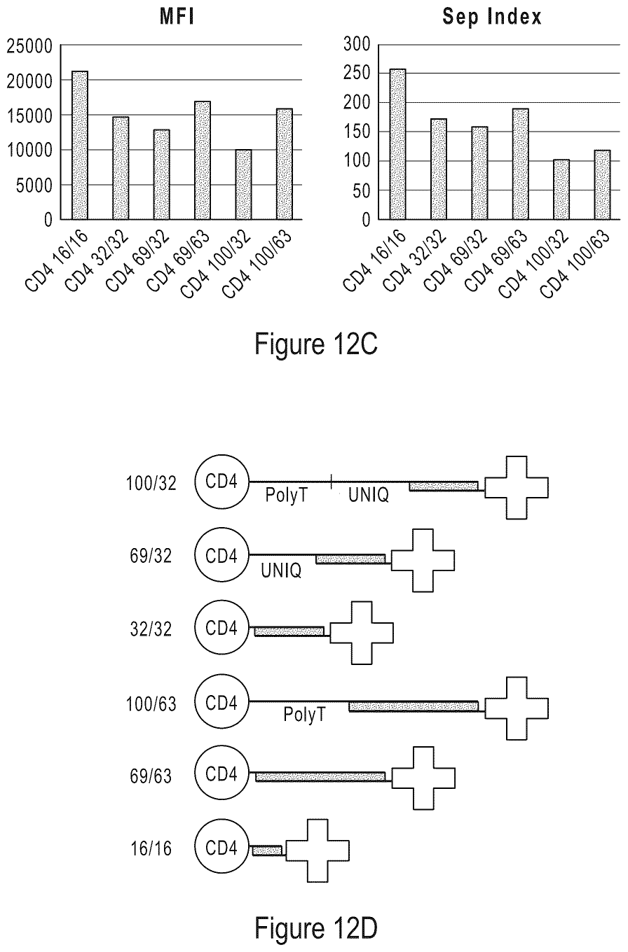

The effect of the nucleic acid linker composition and length of the double-stranded portion was investigated in Example 2 and shown in FIG. 11A through FIG. 13B. A variety of antibody-fluorescent nucleic acid nanostructure conjugates were made and are depicted in FIG. 11B. Some of the conjugates had a short, fully double-stranded linker as seen in FIG. 11B, conjugates 16/16 and 32/32, whereas others had a longer nucleic acid linker that was partially double-stranded as seen in FIG. 11B, conjugates 69/32, 69/63, 100/32 and 100/63. As shown in FIGS. 12A-12D, the composition of the linker strongly influenced the the performance of the conjugates in flow cytometry, as measured by the median fluorescence intensity (MFI). Surprisingly, it was found that the shorter linkers that were fully double-stranded had the best performance, see FIGS. 12A-12D, conjugates 16/16 and 69/63. Intermediate performance was seen with the 32-mer or the partially double-stranded linker with an exposed poly(T) region, see FIGS. 12A-12D, conjugates 32/32 and 100/63. The poorest performance was observed with partially double-stranded linkers with an exposed unique identifying sequence, see FIGS. 12A-12D, conjugates 69/32 and 100/32. Thus, the nucleic acid linker composition and length of the double-stranded portion of the nucleic acid linker can be used to tune the brightness of the polynucleotide-modified antibody bioconjugates and polynucleotide-modified biomolecule bioconjugates provided herein.

Accordingly, provided herein is a method of tuning the brightness of a polynucleotide-modified biomolecule bioconjugate of the present disclosure, the method comprising i) altering the total length of the nucleic acid linker, ii) altering the length of the fully double-stranded region of the nucleic acid linker, iii) altering the length of the single-stranded portion of the nucleic acid linker, and/or iv) having the single-stranded portion comprise a poly(A), poly(T), poly(G), poly(C) sequence and/or a unique nucleic acid sequence. In certain embodiments, a method of increasing the brightness of a polynucleotide-modified biomolecule bioconjugate of the present disclosure is provided, the method comprising, i) decreasing the total length of the nucleic acid linker to 70 nucleotides or fewer, and/or ii) increasing the length of the fully double-stranded region of the nucleic acid linker. In certain embodiments, the nucleic acid linker is fully double-stranded. In certain embodiments, the nucleic acid linker is mostly double-stranded. In certain embodiments, the nucleic acid linker is 70 nucleotides or fewer, 60 nucleotides or fewer, 50 nucleotides or fewer, 40 nucleotides or fewer, 30 nucleotides or fewer, or 20 nucleotides or fewer. In certain embodiments, the nucleic acid linker is between 10 and 70 nucleotides in length, between 10 and 60 nucleotides in length, between 10 and 50 nucleotides in length, between 10 and 40 nucleotides in length, between 10 and 30 nucleotides in length, or between 10 and 20 nucleotides in length.

While not limited to any particular complementary sequences, in certain embodiments, the nucleic acid linker can comprise complementary polyadenosine (poly(A)) and polythymidine (poly(T)) sequences and/or complementary polycytosine (poly(C)) and polyguanidine (poly(G)) sequences. For example, the C:G content of a nucleic acid is known to be a key thermodynamic determinate of double-stranded interactions. In certain embodiments, the double-stranded segment of the nucleic acid linker comprises a poly(A) sequence in one strand and a polythymidine poly(T) sequence in the other strand. In certain embodiments, the double-stranded segment of the nucleic acid linker comprises a poly(C) sequence in one strand and a poly(G) sequence in the other strand. In certain embodiments, the double-stranded segment of the nucleic acid linker comprises poly(A) and poly(C) sequences in one strand and poly(T) and poly(G) sequences in the other strand. In certain embodiments, the double-stranded segment of the nucleic acid linker comprises poly(A) and poly(G) sequences in one strand and poly(T) and poly(C) sequences in the other strand. In certain embodiments, the double-stranded segment of the nucleic acid linker comprises poly(A), poly(T), poly(C), and/or poly(G) sequences in one strand and poly(T), poly(A), poly(G), and/or poly(C) sequences in the other strand. In certain embodiments, the double-stranded segment of the nucleic acid linker consists of a polyadenosine sequence (poly(A)) in one strand and a polythymidine sequence (poly(T)) in the other strand. In certain embodiments, the double-stranded segment of the nucleic acid linker consists of a polycytosine sequence (poly(C)) in one strand and a polyguanidine sequence (poly(G)) in the other strand. One of ordinary skill in the art reading this disclosure will understand that it is contemplated that any nucleic acid linker of any of the embodiments herein can have the above compositions.

### Kits

Also provided for herein are kits for performing any of the multimodal methods of this disclosure. In certain embodiments, the kit comprises a fluorescent-labeled sequence-tagged specificity determining molecule conjugate as described elsewhere herein, or a component or components thereof. In certain embodiments, the kit comprises reagents and/or apparatus for performing cell enrichment, cell sorting, and/or immunofluorescent cell labeling and/or for genomic analysis. In certain embodiments, the kit further comprises instructions either printed and/or on an electronic storage medium, buffers and/or additional reagents, and/or packaging materials.

### Nucleic Acid Nanostructure Fluorescent Labels

Nucleic acid nanostructure fluorescent labels have been described in detail in WO/2018/231805, which is incorporated herein by reference in its entirety. Nucleic acid nanostructure fluorescent labels, which can be used as labels can be created via a variety of techniques. In some examples, DNA self-assembly can be used to ensure that the relative locations of the resonators within a label correspond to locations specified according to a desired temporal decay profile. For example, each resonator of the network could be coupled to a respective specified DNA strand. Each DNA strand could include one or more portions that complement portions one or more other DNA strands such that the DNA strands self-assemble into a nanostructure that maintains the resonators at the specified relative locations.

In certain embodiments the nucleic acid nanostructure fluorescent label comprises one or more polynucleotides. In certain embodiments one or more of those polynucleotides has a length of at least about 10, 15, 20, 25, 30, 35, 40, 50, 55, 60, 65, 75, 80, 85, 90, 95, 100, 110, 120, 130, 140, 150, 175, 200, 300, 400, 500, 750, 1000, 2000, 3000, 4000, 5000, 7500, or 10,000 nucleotides, or any range in between. In certain embodiments one or more of those polynucleotides has a length of at least about 10, 15, 20, 25, 30, 35, 40, 50, 55, 60, 65, 75, 80, 85, 90, 95, 100, 110, 120, 130, 140, 150, 175, 200, 300, or 400 nucleotides, or any range in between. In certain embodiments, one or more of those polynucleotides has a length of at least about 20, 25, 30, 35, 40, 50, 55, 60, 65, 75, 80, 85, 90, 95, 100, 110, or 120 nucleotides, or any range in between. In certain embodiments, the nucleic acid nanostructure fluorescent label comprises two, three, four, five, six or more polynucleotides. In certain embodiments, the nucleic acid nanostructure fluorescent label comprises a total number of nucleotides of at least about 50, 100, 200, 500, 1000, 5000, 10000, 15000, 20000, or any range in between.

DNA self-assembly and other emerging nano-scale manufacturing techniques permit the fabrication of many instances of a specified structure with precision at the nano-scale. For example, as described in WO/2018/231805, a nucleic acid nanostructure, which includes a PHITON nucleic acid nanostructure, is made by annealing custom, synthetic DNA produced by chemical methods. The multiple strands are pre-conjugated to fluorophores, peptides, small molecules, etc. prior to being mixed and annealed. The sequences are designed such that there is a single, finite assembly of lowest energy and is stable in solution, dry, or frozen and preserves the relative location of any conjugated materials. Such precision can permit fluorophores, quantum dots, dye molecules, plasmonic nanorods, or other optical resonators to be positioned at precise locations and/or orientations relative to each other in order to create a variety of optical resonator networks. Such resonator networks may be specified to facilitate a variety of different applications. In some examples, the resonator networks could be designed such that they exhibit a pre-specified temporal relationship between optical excitation (e.g., by a pulse of illumination) and re-emission; this could enable temporally-multiplexed labels and taggants that could be detected using a single excitation wavelength and a single detection wavelength. Additionally, or alternatively, the probabilistic nature of the timing of optical re-emission, relative to excitation, by these resonator networks could be leveraged to generate samples of a random variable. These resonator networks may include one or more “input resonators” that exhibit a dark state; resonator networks including such input resonators may be configured to implement logic gates or other structures to control the flow of excitons or other energy through the resonator network. Such structures could then be used, e.g., to permit the detection of a variety of different analytes by a single resonator network, to control a distribution of a random variable generated using the resonator network, to further multiplex a set of labels used to image a biological sample, or to facilitate some other application.

These resonator networks include networks of fluorophores, quantum dots, dyes, Raman dyes, conductive nanorods, chromophores, or other optical resonator structures. The networks can additionally include antibodies, aptamers, strands of deoxyribonucleic acid (DNA) or ribonucleic acid (RNA), or other receptors configured to permit selective binding to analytes of interest (e.g., to a surface protein, molecular epitope, characteristic nucleotide sequence, or other characteristic feature of an analyte of interest). The labels can be used to observe a sample, to identify contents of the sample (e.g., to identity cells, proteins, or other particles or substances within the sample), to sort such contents based on their identification (e.g., to sort cells within a flow cytometer according to identified ceil type or other properties), or to facilitate some other applications. In certain embodiments disclosed herein the labels are linked to a substrate, such as an antibody or bead, via a polynucleotide linker.

In an example application, such resonator networks may be applied (e.g., by-coupling the resonator network to an antibody, aptamer, or other analyte-specific receptor) to detect the presence of, discriminate between, or otherwise observe a large number of different labels in a biological or material sample or other environment of interest. Such labels may permit detection of the presence, amount, or location of one or more analytes of interest in a sample (e.g., in a channel of a flow cytometry apparatus). Having access to a large library of distinguishable labels can allow for the simultaneous detection of a large number of different analytes. Additionally, or alternatively, access to a large library of distinguishable labels can allow for more accurate detection of a particular analyte (e.g., a cell type or sub-type of interest) by using multiple labels to bind with the same analyte, e.g., to different epitopes, surface proteins, or other features of the analyte. Yet further, access to such a large library of labels may permit selection of labels according to the probable density or number of corresponding analytes of interest, e.g., to ensure that the effective brightness of different labels, corresponding to analytes having different concentrations in a sample, is approximately the same when optically interrogating such a sample.

Such labels may be distinguishable by virtue of differing with respect to an excitation spectrum, an emission spectrum, a fluorescence lifetime, a fluorescence intensity, a susceptibility to photobleaching, a fluorescence dependence on binding to an analyte or on some other environmental factor, a polarization of re-emitted light, or some other optical properties.

WO/2018/231805 describes methods for specifying, fabricating, detecting, and identifying optical labels that differ with respect to temporal decay profile and/or excitation and emission spectra. Additionally, or alternatively, the provided labels may have enhanced brightness relative to existing labels (e.g., fluorophore-based labels) and may have a configurable brightness to facilitate panel design or to permit the relative brightness of different labels to facilitate some other consideration. Such labels can differ with respect to the time-dependent probability of re-emission of light by the label subsequent to excitation of the label (e.g., by an ultra-fast laser pulse). Additionally, or alternatively, such labels can include networks of resonators to increase a difference between the excitation wavelength of the labels and the emission wavelength of the labels (e.g., by interposing a number of mediating resonators between an input resonator and an output resonator to permit excitons to be transmitted between input resonators and output resonators between which direct energy-transfer is disfavored). Yet further, such labels may include logic gates or other optically-controllable structures to permit further multiplexing when detecting and identifying the labels.

Resonator networks (e.g., resonator networks included as part of labels) as described in WO/2018/231805 can be fabricated in a variety of ways such that one or more input and/or readout resonators, output resonators, dark-state-exhibiting “logical input” resonators, and/or mediating resonators are arranged according to a specified network of resonators and further such that a temporal decay profile of the network, a brightness of the network, an excitation spectrum, an emission spectrum, a Stokes shift, or some other optical property of the network, or some other detectable property of interest of the network (e.g. , a state of binding to an analyte of interest) corresponds to a specification thereof (e.g., to a specified temporal decay profile, a probability of emission in response to illumination). Such arrangement can include ensuring that a relative location, distance, orientation, or other relationship between the resonators (e.g., between pairs of the resonators) correspond to a specified location, distance, orientation, or other relationship between the resonators.

This can include using DNA self-assembly to fabricate a plurality of instances of one or more resonator networks. For example, a number of different DNA strands could be coupled (e.g., via a primary amino modifier group on thymidine to attach an N-Hydroxysuccinimide (NHS) ester-modified dye molecule) to respective resonators of a resonator networks (e.g., input resonators, output resonator, and/or mediator resonators). Pairs of the DNA strands could have portions that are at least partially complementary such that, when the DNA strands are mixed and exposed to specified conditions (e.g., a specified pH, or a specified temperature profile), the complementary portions of the DNA strands align and bind together to form a semi-rigid nanostructure that maintains the relative locations and/or orientations of the resonators of the resonator networks.

In a representative resonator network, an input resonator, an output resonator and two mediator resonators are coupled to respective DNA strands. The coupled DNA strands, along with additional DNA strands, then self-assemble into the illustrated nanostructure such that the input resonator, mediator resonators, and output resonator form a resonator wire. In some examples, a plurality of separate identical or different networks could be formed, via such methods or other techniques, as part of a single instance of a resonator network (e.g., to increase a brightness of the resonator network).

The distance between resonators of such a resonator network could be specified such that the resonator network exhibits one or more desired behaviors (e.g., is excited by light at a particular excitation wavelength and responsively re-emits light at an emission wavelength according to a specified temporal decay profile). This can include specifying the distances between neighboring resonators such that they are able to transmit energy between each other (e.g., bidirectionally or unidirectionally) and further such that the resonators do not quench each other or otherwise interfere with the optical properties of each other. In examples wherein the resonators are bound to a backbone via linkers (e.g., to a DNA backbone via an amide bond (created, e.g., by N-Hydroxysuccinimide (NHS) ester molecules) or other linking structures), the linkers can be coupled to locations on the background that are specified with these considerations, as well as the length(s) of the linkers, in mind. For example, the coupling locations could be separated by a distance that is more than twice the linker length (e.g., to prevent the resonators from coming into contact with each other, and thus quenching each other or otherwise interfering with the optical properties of each other). Additionally, or alternatively, the coupling locations could be separated by a distance that is less than a maximum distance over which the resonators may transmit energy between each other. For example, the resonators could be fluorophores or some other optical resonator that is characterized by a Förster radius when transmitting energy via Förster resonance energy transfer, and the coupling locations could be separated by a distance that is less than the Förster radius.

### EXAMPLES

### Example 1

The cell surface protein expression of an individual cell is its identity. For example, memory CD4+ T cells, there is a certain subset that expresses CCR7. While this is a definition of identity (CD3+CD4+CD8−CCR7+), this is also linked inherently to the function of these cells: CD4 is a co-receptor for the T cell receptor that recognizes the MHCII complex, CD3 is part of the signaling complex for the T cell receptor, and CCR7 enables the CD4+ T cell to migrate towards areas of higher concentration of CCL19/21 expression. This expression, in turn, is higher in lymph nodes and higher still in the B cell areas within. Thus, a simple expression of cell identity, of which there are hundreds in the immune system, and many thousands besides when considering a whole organism, contains a wealth of information. Additionally, this identity is their sorting definition (for cell enrichment, which is a critical step in single cell sequencing) and their identity in imaging.

In contrast, when one examines the gene expression of those identifying genes, e.g. the expression of CD3, CD4, and CCR7 at the level of RNA, one finds that they are not expressed or expressed in exceedingly low quantities, especially within resting immune cells. As a result, gold standard tools for identifying immune cells in single cell sequencing based on their RNA alone (Seurat, https://satijalab.org/seurat/: Butler, A., Hoffman, P., Smibert, P. et al. Integrating single-cell transcriptomic data across different conditions, technologies, and species. Nat Biotechnol 36, 411-420 (2018). https://doi.org/10.1038/nbt.4096 & Stuart et. al. Comprehensive Integration of Single-Cell Data. Cell. 2019) are based on a handful of genes (generally, less than 10), despite sequencing occurring on all 10,000 protein encoding genes in the case of whole transcriptome analysis (WTA). Additionally, lymphocytes do not express many genes at high levels, leading to a large number of genes measured “living” at the near zero (dropout) range. Thus, by using the fluorescently labeled sequence-tagged specificity determining molecules as provided herein, one can obtain information pertaining to the cell's identity (via cell sorting and/or imaging) as well as the cell's genomic profile or gene expression (via next gen sequencing (e.g., scRNAseq) and/or spatial transcriptomics).

FIG. 9B illustrates a workflow for obtaining cell surface protein and transcript data from individual cells according to certain methods of the present disclosure. Cells (e.g., peripheral blood mononuclear cells (PBMCs)) are stained in suspension with the fluorescently labeled sequence-tagged antibodies provided herein to delineate major immune cell types according to known methods such as Drop-seq or CITE-seq (see for example Stoeckius et al, Nature Methods, 14:865 (2017) and the Supplementary Protocol for a step-by-step protocol for CITE-Seq) or the 10X Genomics Chromium instrument (see for example, “Chromium Next GEM Single Cell 3′ Reagent Kits v3.1 (Dual Index) user guide from 10X Genomics at the world wide web at https://support.10xgenomics.com/single-cell-gene-expression/index/doc/user-guide-chromium-single-cell-3-reagent-kits-user-guide-v31-chemistry-dual-index). Next, fluorescence-activated cell sorting using a cell sorter such as the BIGFOOT Cell Sorter (Thermo Fisher Scientific) is performed to enrich for CD3⁺CD8⁺ T cells and Dendritic cells (CD3⁻CD19⁻CD16⁻CD11c⁺) according to the manufacturer's protocol to enrich for both cell types. Enriched cells are counted, and the cell viability is checked using the COUNTESS 3 FL Automated Cell Counter (Thermo Fisher Scientific) according to the manufacturer's protocol. Ideally, input cell suspensions should contain more than 90% viable cells. If performing scRNAseq using the 10X Genomics Chromium instrument (10X Genomics, Pleasanton, CA), see the manufacturer's protocols for detailed information (at the world wide web at https://support.10xgenomics.com/single-cell-gene-expression/sample-prep/doc/demonstrated-protocol-single-cell-protocols-cell-preparation-guide). Single cell partitioning is then performed. This can be accomplished using various technologies, such as the 10X Genomics Chromium instrument. Once single cell partitioning has been completed, users will have to perform DNA sequencing on an Illumina platform (Illumina, Inc., San Diego, CA) to obtain cell surface protein and transcript data from individual cells. Finally, multimodal (cell surface protein and transcript) analysis of data can be performed using open-source analysis software such as Seurat (Hao et al, “Integrated analysis of multimodal single-cell data,” Cell: 184, 3573-3587 (2021)).

FIG. 10B shows a workflow for obtaining spatial proteogenomics data according to certain methods of the present disclosure. Stored tissue blocks (either formalin-fixed paraffin-embedded (FFPE) or formalin-fixed (FF)) are prepared using standard protocols. A generalized protocol is described here, starting with the tissue already preserved. Using a microtome, slice the tissue and mount onto a charged slide according to standard protocols. Antigen retrieval is performed using methods that will vary by tissue type and application, see for example the VISIUM Spatial Gene Expression platform from 10x Genomics (at the world wide web at https://support.10xgenomics.com/spatial-gene-expression/sample-prep/doc/demonstrated-protocol-visium-spatial-protocols-tissue-preparation-guide), the MERSCOPE platform from Vizgen (at the world wide web at https://vizgen.com/wp-content/uploads/2021/10/91600002_MERSCOPE-Fresh-and-Fixed-Frozen-Tissue-Sample-Preparation-User-Guide.pdf) and immunofluorescence staining according to standard protocols. Tissue samples are stained with the fluorescently labeled sequence-tagged antibodies provided herein. Once the samples are stained, proceed to desired downstream imaging and processing and perform data analysis.

### Example 2

An optimized method for modifying antibodies with single-stranded DNA (ssDNA) can be used to attach oligos of varying lengths. This method entails optimized chemistry to control the degree of labeling of the ssDNA linker, as well as purification methods to remove excess ssDNA linker and unlabeled antibody. FIG. 11A through FIG. 13B show data obtained exploring four different lengths of ssDNA linker attached to anti-human CD4 antibody (clone SK3). A complementary ssDNA linker sequence was incorporated into a fluorescent nucleic acid nanostructure (e.g., a PHITON nucleic acid nanostructure) during folding that could hybridize to all or a portion of the ssDNA linker sequence on the antibody. FIG. 11B shows how a small subset of linker lengths on the antibody and the fluorescent nucleic acid nanostructure were combined in different ways to give six different antibody-fluorescent nucleic acid nanostructure conjugates with varying lengths of double- and single-stranded linkage. FIG. 11C shows a PAGE gel of the antibody conjugates with varying lengths of nucleic acid linker after purification. When tested in flow cytometry (FIGS. 12A-12D) using conjugates of anti-human CD4 antibody (clone SK3) and NOVAFLUOR Yellow 610 (the fluorescent nucleic acid nanostructure) to stain human peripheral blood cells (PBMCs), the varying combinations of linkers influenced the brightness of the signal detected. Such a method illustrated how fluorescent nucleic acid nanostructures can be used to explore the effects of length and sequence on epitope binding for the fluorescent nucleic acid nanostructure labeled sequence-tagged antibody.

FIG. 11A shows antibodies and fluorescent nucleic acid nanostructures (e.g., PHITON nucleic acid nanostructures) that were modified with varying lengths of ssDNA linkers that completely or partially hybridized to one another. FIG. 11B shows the various conjugates of antibody and fluorescent nucleic acid nanostructures using different combinations of the individual components shown in FIG. 11A. FIG. 11C shows a polyacrylamide gel electrophoresis (PAGE) gel showing antibody-ssDNA linker conjugates for each of the four lengths of ssDNA linker on the antibody (16, 32, 69, 100 nucleotides) after purification to remove unmodified antibody.

FIG. 12A shows flow cytometry data from human PBMCs testing the various possible combinations of linkers for attaching a fluorescent nucleic acid nanostructure (in this example NOVAFLUOR Yellow 610) to anti-Human CD4 (SK3) antibody. All conjugates were compared at the same dose. FIGS. 12B and 12C show analysis of the flow cytometry data that compared the median fluorescence intensity (MFI) of the CD4+ population and the separation indices (SI) of the various antibody-NOVAFLUOR Yellow 610 conjugates. The composition of the nucleic acid linker strongly influenced the performance of the conjugate in flow cytometry. FIG. 12D shows the composition of the nucleic acid linkers for each of the conjugates, specifically whether the linker was partially or fully double-stranded and whether the single-stranded portion of the nucleic acid linker contained a poly(T) region and/or a unique identifying sequence (UNIQ).

The effect of the nucleic acid linker composition and length of the double-stranded portion was investigated in FIG. 11A through FIG. 13B. A variety of antibody-fluorescent nucleic acid nanostructure conjugates were made and are depicted in FIG. 11B. Some of the conjugates had a short, fully double-stranded nucleic acid linker as seen in FIG. 11B, conjugates 16/16 and 32/32, whereas others had a longer nucleic acid linker that was partially double-stranded as seen in FIG. 11B, conjugates 69/32, 69/63, 100/32 and 100/63. As shown in FIGS. 12A-12D, the composition of the nucleic acid linker strongly influenced the fluorescence intensity (MFI) of the conjugates as well as the performance of the conjugates in flow cytometry. Surprisingly, it was found that the shorter nucleic acid linkers that were fully double-stranded had the best performance, see FIGS. 12A-12D, conjugates 16/16 and 69/63. Intermediate performance was seen with the 32-mer or the partially double-stranded nucleic acid linker with an exposed poly(T) region, see FIGS. 12A-12D, conjugates 32/32 and 100/63. The poorest performance was observed with partially double-stranded nucleic acid linkers with an exposed unique identifying sequence, see FIGS. 12A-12D, conjugates 69/32 and 100/32.

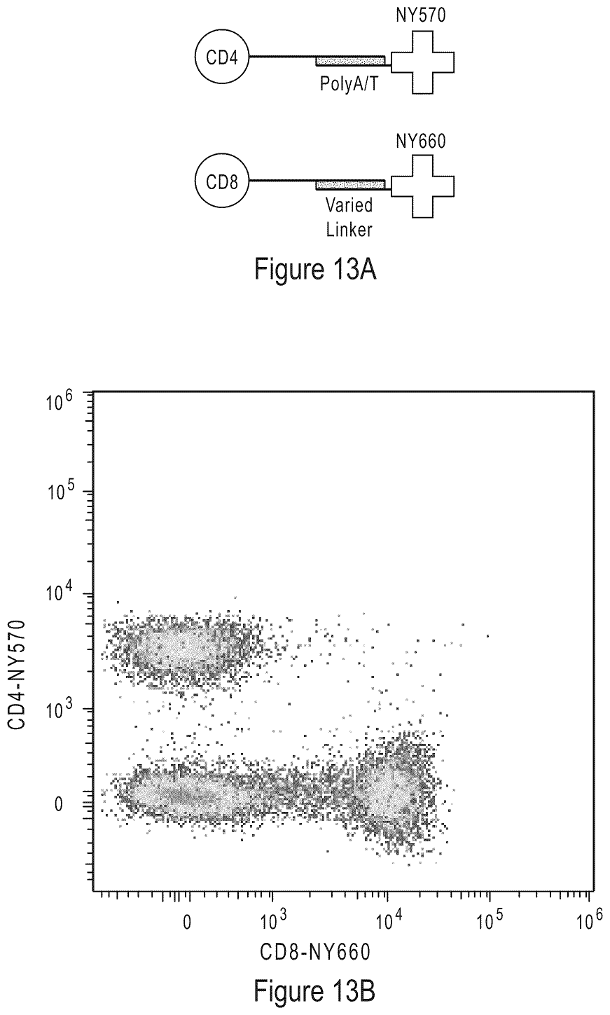

Separately, DNA linkers with distinct sequences were tested with different antibody-fluorescent nucleic acid nanostructure conjugates to illustrate that more than one of these reagents can be used together in the same multiplexed experiment. FIGS. 13A-13B show anti-human CD4 antibody (clone SK3) conjugated to NOVAFLUOR Yellow 570 and anti-human CD8 antibody (clone OKT-8) conjugated to NOVAFLUOR Yellow 660 assembled with a poly(A)/poly (T) linker (CD4 conjugate) and a more varied nucleic acid linker sequence (CD8 conjugate). These conjugates were used together to distinctly stain their target populations on human PBMCs with no cross-reactivity. Such a strategy could be extended to other unique DNA sequences and illustrates the feasibility of using many of these conjugates together in the workflows discussed.

The breadth and scope of the present disclosure should not be limited by any of the above-described exemplary embodiments but should be defined only in accordance with the following claims and their equivalents.

1. A method for combining cell enrichment, cell sorting, and/or immunofluorescent cell labeling with genomic analysis using a sequence-tagged fluorescent-label specificity determining molecule conjugate comprising a fluorescent label component and a specificity determining molecule component, wherein one or more components of the conjugate are used for cell enrichment, cell sorting, and/or immunofluorescent cell labeling and one or more components of the same conjugate are utilized in the genomic analysis;
the method comprising (a) performing cell enrichment, cell sorting, and/or immunofluorescent cell labeling on a cell and/or sample of cells and (b) performing genomic analysis on the same cell and/or sample of cells, using the fluorescent-labeled sequence-tagged specificity determining molecule conjugate;
wherein the fluorescent label component is a fluorescently labeled nucleic acid nanostructure;
wherein the specificity determining molecule component is sequence-tagged;
wherein the method first comprises contacting the cell and/or sample of cells with the fluorescent-labeled sequence-tagged specificity determining molecule conjugate;
wherein the genomic analysis occurs after the cell enrichment, cell sorting, and/or immunofluorescent cell labeling; and
wherein the specificity determining molecule is an antibody or an antigen-binding fragment, variant, or derivative thereof.

2. The method of claim 1, wherein the fluorescent label component is attached to the specificity determining molecule component via a nucleic acid linker;
wherein the nucleic acid linker comprises a double-stranded segment; or
wherein the nucleic acid linker is entirely double-stranded.

3. (canceled)

4. The method of claim 1, wherein the nucleic acid linker is double-stranded and is between 10 and 70 nucleotides.

5. The method of claim 1, wherein the specificity determining molecule component comprises a PCR primer region, a barcode region and a capture sequence.

6-7. (canceled)

8. The method of claim 1,
wherein the choice of cell enrichment, cell sorting, and/or immunofluorescent cell labeling method is not limiting on the choice of genomic analysis method; or
wherein the choice of genomic analysis method is based on the results of the cell enrichment, cell sorting, and/or immunofluorescent cell labeling.

9. The method of claim 1,
wherein the cell enrichment and/or cell sorting comprises flow cytometry/FACS; and/or
wherein the fluorescent cell labeling comprises visualization and/or quantitation such as with single- or multi-photon microscopy, intravital microscopy, super resolution microscopy, whole tissue imaging, traditional fluorescence microscopy (IF-IC (immunocytochemistry)), IF-F (frozen), and/or mIHC (multiplexed immunohistochemistry).

10. The method of claim 1, wherein the genomic analysis comprises Sanger sequencing, next generation sequencing (NGS), long-read sequencing, in situ sequencing, PCR, and/or RT-PCR.

11. The method of claim 1, wherein the method is applied to a single-cell suspension, bulk tissue measurement, and/or an imaging application.

12-21. (canceled)

22. The method of claim 1,
wherein the degree of labeling (DoL) of the specificity determining molecule component is used to increase the signal detection; and/or
wherein the fluorescent label is a fluorescently labeled nucleic acid nanostructure and the number of fluorescent molecules incorporated into the fluorescently labeled nucleic acid nanostructure is used to increase the signal detection.

23-25. (canceled)

26. The method of claim 1, wherein the use of a sequence-tagged fluorescent-label specificity determining molecule conjugate allows data from cell enrichment, cell sorting, and/or immunofluorescent cell labeling to be linked to data from genomic analysis.

27. A sequence-tagged fluorescent-label specificity determining molecule conjugate comprising a specificity determining molecule component conjugated to a fluorescent label component, wherein said conjugate is suitable for use in the method of claim 1;
wherein the fluorescent label component is a fluorescently labeled nucleic acid nanostructure;
wherein the specificity determining molecule component is sequence-tagged; and
wherein the specificity determining molecule is an antibody or an antigen-binding fragment, variant, or derivative thereof.

28. The conjugate of claim 27, wherein the fluorescent label component is attached to the specificity determining molecule component via a nucleic acid linker;
wherein the nucleic acid linker comprises a double-stranded segment; or
wherein the nucleic acid linker is entirely double-stranded.

29. (canceled)

30. The conjugate of claim 28, wherein the nucleic acid linker is double-stranded and is between 10 and 70 nucleotides.

31. The conjugate of claim 27, wherein the specificity determining molecule component comprises a PCR primer region, a barcode region and a capture sequence.

32-39. (canceled)

40. The conjugate of claim 27, wherein the conjugate comprises one or more unique identifying sequence.

41. The conjugate of claim 27, wherein the specificity determining molecule component is linked to the fluorescent label component by a nucleic acid linker, wherein the nucleic acid linker is single-stranded, at least partially double-stranded, or entirely double-stranded; or
wherein the nucleic acid linker is a hybridized at least partially double-stranded nucleic acid, the specificity determining molecule component is covalently attached to one strand of the linker, the fluorescent label component is covalently attached to the opposite strand of the linker, and wherein the specificity determining molecule component and the fluorescent label component are not covalently attached but instead linked via the hybridization of their respective linker strands.

42. The conjugate of claim 28, wherein the nucleic acid linker is a hybridized entirely double-stranded nucleic acid, the specificity determining molecule component is covalently attached to one strand of the linker, the fluorescent label component is covalently attached to the opposite strand of the linker, and wherein the specificity determining molecule component and the fluorescent label component are not covalently attached but instead linked via the hybridization of their respective linker strands.

43-46. (canceled)

47. A kit comprising,
the sequence-tagged fluorescent-label specificity determining molecule conjugate of claim 27, or a component thereof;
one or more reagents for performing cell enrichment, cell sorting, or immunofluorescent cell labeling, and genomic analysis; and
instructions either printed and/or on an electronic storage medium, buffers and/or additional reagents, and/or packaging materials.

48. (canceled)

49. A method of increasing the brightness of a sequence-tagged fluorescent-label specificity determining molecule conjugate, the method comprising,
i) decreasing the total length of the nucleic acid linker to 50 nucleotides or less, and/or
ii) increasing the length of the fully double-stranded region of the nucleic acid linker.

50-52. (canceled)

53. A method of tuning the brightness of a sequence-tagged fluorescent-label specificity determining molecule conjugate of claim 27, the method comprising:
i) altering the total length of the nucleic acid linker;
ii) altering the length of the fully double-stranded region of the nucleic acid linker;
iii) altering the length of the single-stranded portion of the nucleic acid linker; and/or
iv) having the single-stranded portion comprise a poly(A), poly(T), poly(G), poly(C) sequence and/or a unique nucleic acid sequence.


SEQUENCE LISTING (3 sequences)

SEQ ID NO: 1 - DNA, 29 bp, Artificial Sequence
  source: /note=“Description of Artificial Sequence Synthetic oligonucleotide”
aaaaaaaaaaaaaaaaaaaaaaaaaaaaa

SEQ ID NO: 2 - DNA, 10 bp, Artificial Sequence
  source: /note=“Description of Artificial Sequence Synthetic oligonucleotide”
gagcgaatcg

SEQ ID NO: 3 - DNA, 10 bp, Artificial Sequence
  source: /note=“Description of Artificial Sequence Synthetic oligonucleotide”
cgattcgctc